(19) United States
(12) Patent Application Publication
(10) Pub. No.: US 2005/0182061 A1
(43) Pub. Date: Aug. 18, 2005

(54) Phthalimide compounds useful as protein kinase inhibitors

(76) Inventors: Jeremy Green, Burlington, MA (US); Craig Marhefka, Rockville, MD (US)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 10/958,167

(22) Filed: Oct. 4, 2004

Related U.S. Application Data
(60) Provisional application No. 60/508,499, filed on Oct. 2, 2003.

(51) Int. Cl. C07D 471/02; A61K 31/498; A61K 31/4745; C07D 487/02; A61K 31/4035

(52) U.S. Cl. 514/249; 514/300; 514/417; 544/350; 546/113; 548/473

(57) ABSTRACT

The present invention provides compounds as inhibitors of protein kinase, particularly inhibitors of AKT, PDK1, p70S6K, or ROCK kinase, mammalian protein kinases involved in proliferative and neurodegenerative disorders. The invention also provides processes for preparing the compounds of the invention, pharmaceutical compositions comprising the compounds, and methods of utilizing those compounds and compositions in the treatment of various disorders.

### CROSS REFERENCE TO RELATED APPLICATIONS

The present application claims the benefit, under 35 U.S.C. § 119, of U.S. Provisional patent application No. 60/508,499, filed Oct. 2, 2003, entitled “Phthalimide Compounds Useful as Protein Kinase Inhibitors”, the entire contents of which are hereby incorporated by reference.

### FIELD OF THE INVENTION

The present invention is in the field of medicinal chemistry and relates to compounds that are protein kinase inhibitors, methods for making such compounds, compositions containing such compounds and methods of use. More particularly, the compounds are inhibitors of the AGC family of protein kinases including, but not limited to, AKT, PDK1, p70S6K, and ROCK kinases and are useful for treating diseases, such as cancer.

### BACKGROUND OF THE INVENTION

The search for new therapeutic agents has been greatly aided in recent years by better understanding of the structure of enzymes and other biomolecules associated with target diseases. One important class of enzymes that has been the subject of extensive study is the protein kinases.

Protein kinases mediate intracellular signal transduction. They do this by effecting a phosphoryl transfer from a nucleoside triphosphate to a protein acceptor that is involved in a signaling pathway. There are a number of kinases and pathways through which extracellular and other stimuli cause a variety of cellular responses to occur inside the cell. Examples of such stimuli include environmental and chemical stress signals (e.g. osmotic shock, heat shock, ultraviolet radiation, bacterial endotoxin, H₂O₂), cytokines (e.g. interleukin-1 (IL-1) and tumor necrosis factor α (TNF-α)), and growth factors (e.g. granulocyte macrophage-colony-stimulating factor (GM-CSF), and fibroblast growth factor (FGF). An extracellular stimulus may effect one or more cellular responses related to cell growth, migration, differentiation, secretion of hormones, activation of transcription factors, muscle contraction, glucose metabolism, control of protein synthesis and regulation of cell cycle.

Many diseases are associated with abnormal cellular responses triggered by protein kinase-mediated events. These diseases include autoimmune diseases, inflammatory diseases, neurological and neurodegenerative diseases, cancer, cardiovascular diseases, allergies and asthma, Alzheimer's disease or hormone-related diseases. Accordingly, there has been a substantial effort in medicinal chemistry to find protein kinase inhibitors that are effective as therapeutic agents. A challenge has been to find protein kinase inhibitors that act in a selective manner. Since there are numerable protein kinases that are involved in a variety of cellular responses, non-selective inhibitors may lead to unwanted side effects.

The AGC sub-family of kinases phosphorylate their substrates at serine and threonine residues and participate in a variety of well-known signaling processes, including, but not limited to cyclic AMP signaling, the response to insulin, apoptosis protection, diacylglycerol signaling, and control of protein translation (Peterson et al., Curr. Biol. 1999, 9, R521). This sub-family includes PKB (c-Akt), PKC, PRK1, 2, p70S6K, and PDK.

AKT (also known as PKB or Rac-PK beta), a serine/threonine protein kinase, has been shown to be overexpressed in several types of cancer and is a mediator of normal cell functions [(Khwaja, A., Nature 1999, 401, 33-34); (Yuan, Z. Q., et al., Oncogene 2000, 19, 2324-2330); (Namikawa, K., et al., J Neurosci. 2000, 20, 2875-2886,)]. AKT comprises an N-terminal pleckstrin homology (PH) domain, a kinase domain and a C-terminal “tail” region. Three isoforms of human AKT kinase (AKT-1, -2 and -3) have been reported so far [(Cheng, J. Q., Proc. Natl. Acad. Sci. USA 1992, 89, 9267-9271); (Brodbeck, D. et al., J. Biol. Chem. 1999, 274, 9133-9136)]. The PH domain binds 3-phosphoinositides, which are synthesized by phosphatidyl inositol 3-kinase (PI3K) upon stimulation by growth factors such as platelet derived growth factor (PDGF), nerve growth factor (NGF) and insulin-like growth factor (IGF-1) [(Kulik et al., Mol. Cell. Biol., 1997, 17, 1595-1606,); (Hemmings, B. A., Science, 1997, 275, 628-630)]. Lipid binding to the PH domain promotes translocation of AKT to the plasma membrane and facilitates phosphorylation by another PH-domain-containing protein kinases, PDK1 at Thr308, Thr309, and Thr305 for the AKT isoforms 1, 2 and 3, respectively. A second, as of yet unknown, kinase is required for the phosphorylation of Ser473, Ser474 or Ser472 in the C-terminal tails of AKT-1, -2 and -3 respectively, in order to yield a fully activated AKT enzyme.

Once localized to the membrane, AKT mediates several functions within the cell including the metabolic effects of insulin (Calera, M. R. et al., J. Biol. Chem. 1998, 273, 7201-7204) induction of differentiation and/or proliferation, protein synthesis and stress responses (Alessi, D. R. et al., Curr. Opin. Genet. Dev. 1998, 8, 55-62).

Manifestations of altered AKT regulation appear in both injury and disease, the most important role being in cancer. The first account of AKT was in association with human ovarian carcinomas where expression of AKT was found to be amplified in 15% of cases (Cheng, J. Q. et al., Proc. Natl. Acad. Sci. U.S.A. 1992, 89, 9267-9271). It has also been found to be overexpressed in 12% of pancreatic cancers (Cheng, J. Q. et al., Proc. Natl. Acad. Sci. U.S.A. 1996, 93, 3636-3641). It was demonstrated that AKT-2 was over-expressed in 12% of ovarian carcinomas and that amplification of AKT was especially frequent in 50% of undifferentiated tumours, suggesting that AKT may also be associated with tumour aggressiveness (Bellacosa, et al., Int. J. Cancer 1995, 64, 280-285).

The ribosomal protein kinases p70S6K-1 and -2 are members of the AGC sub-family of protein kinases that consists of, amongst others, PKB and MSK. The p70S6 kinases catalyze the phosphorylation and subsequent activation of the ribosomal protein S6, which has been implicated in the translational up-regulation of mRNAs coding for the components of protein synthetic apparatus.

These mRNAs contain an oligopyrimidine tract at their 5′ transcriptional start site, termed a 5′TOP, which has been shown to be essential for their regulation at the translational level (Volarevic, S. et al., Prog. Nucleic Acid Res. Mol. Biol. 65, pp 101-186, 2001). p70 S6K dependent S6 phosphorylation is stimulated in response to a variety of hormones and growth factors primarily via the PI3K pathway (Coffer, P. J. et al., Biochem. Biophys. Res. Commun, 198, 7 pp 780-786, 1994), which maybe under the regulation of mTOR, since rapamycin acts to inhibit p70S6K activity and blocks protein synthesis, specifically as a result of a down-regulation of translation of these mRNA's encoding ribosomal proteins (Kuo, C. J. et al., Nature, 358, pp 70-73, 1992).

In vitro PDK1 catalyses the phosphorylation of Thr252 in the activation loop of the p70 catalytic domain, which is indispensable for p70 activity (Alessi, D. R., Curr. Biol., 8, pp 69-81, 1998). The use of rapamycin and gene deletion studies of dp70S6K from Drosophila and p70S6K1 from mouse have established the central role p70 plays in both cell growth and proliferation signaling.

The 3-phosphoinositide-dependent protein kinase-1 (PDK1) plays a key role in regulating the activity of a number of kinases belonging to the AGC subfamily of protein kinases (Alessi, D. et al., Biochem. Soc. Trans, 29, pp. 1, 2001). These include isoforms of protein kinase B (PKB, also known as AKT), p70 ribosomal S6 kinase (S6K) (Avruch, J. et al., prog. Mol. Subcell. Biol., 2001, 26, pp. 115, 2001), and p90 ribosomal S6 kinase (Frödin, M. et al., EMBO J., 19, pp. 2924-2934, 2000). PDK1 mediated signaling is activated in response to insulin and growth factors and as a consequence of attachment of the cell to the extracellular matrix (integrin signaling). Once activated these enzymes mediate many diverse cellular events by phosphorylating key regulatory proteins that play important roles controlling processes such as cell survival, growth, proliferation and glucose regulation [(Lawlor, M. A. et al., J. Cell Sci., 114, pp. 2903-2910, 2001), (Lawlor, M. A. et al., EMBO J., 21, pp. 3728-3738, 2002)]. PDK1 is a 556 amino acid protein, with an N-terminal catalytic domain and a C-terminal pleckstrin homology (PH) domain, which activates its substrates by phosphorylating these kinases at their activation loop (Belham, C. et al., Curr. Biol., 9, pp. R93-R96, 1999). Many human cancers including prostate and NSCL have elevated PDK1 signaling pathway function resulting from a number of distinct genetic events such as PTEN mutations or over-expression of certain key regulatory proteins [(Graff, J. R., Expert Opin. Ther. Targets, 6, pp. 103-113, 2002), (Brognard, J., et al., Cancer Res., 61, pp. 3986-3997, 2001)]. Inhibition of PDK1 as a potential mechanism to treat cancer was demonstrated by transfection of a PTEN negative human cancer cell line (U87MG) with antisense oligonucleotides directed against PDK1. The resulting decrease in PDK1 protein levels led to a reduction in cellular proliferation and survival (Flynn, P., et al., Curr. Biol., 10, pp. 1439-1442, 2000). Consequently the design of ATP binding site inhibitors of PDK1 offers, amongst other treatments, an attractive target for cancer chemotherapy.

The diverse range of cancer cell genotypes has been attributed to the manifestation of the following six essential alterations in cell physiology: self-sufficiency in growth signaling, evasion of apoptosis, insensitivity to growth-inhibitory signaling, limitless replicative potential, sustained angiogenesis, and tissue invasion leading to metastasis (Hanahan, D. et al., Cell, 100, pp. 57-70, 2000). PDK1 is a critical mediator of the PI3K signalling pathway, which regulates a multitude of cellular function including growth, proliferation and survival. Consequently inhibition of this pathway could affect four or more of the six defining requirements for cancer progression, as such it is anticipated that a PDK1 inhibitor will have an effect on the growth of a very wide range of human cancers.

Specifically, increased levels of PI3K pathway activity has been directly associated with the development of a number of human caners, progression to an aggressive refractory state (acquired resistance to chemotherapies) and poor prognosis. This increased activity has been attributed to a series of key events including decreased activity of negative pathway regulators such as the phosphatase PTEN, activating mutations of positive pathway regulators such as Ras, and overexpression of components of the pathway itself such as PKB, examples include: brain (gliomas), breast, colon, head and neck, kidney, lung, liver, melanoma, ovarian, pancreatic, prostate, sarcoma, thyroid [(Teng, D. H. et al., Cancer Res., 57, pp. 5221-5225, 1997), (Brognard, J. et al., Cancer Res., 61, pp. 3986-3997, 2001), (Cheng, J. Q. et al., Proc. Natl. Acad. Sci., 93, pp. 3636-3641, 1996), Int. J. Cancer, 64, pp. 280, 1995), (Graff, J. R., Expert Opin. Ther. Targets, 6, pp. 103-113, 2002), Am. J. Pathol., 159, pp. 431, 2001)].

Additionally, decreased pathway function through gene knockout, gene knockdown, dominant negative studies and small molecule inhibitors of the pathway have been demonstrated to reverse many of the cancer phenotypes in vitro (some studies have also demonstrated a similar effect in vivo) such as block proliferation, reduce viability and sensitize cancer cells to known chemotherapies in a series of cell lines, representing the following cancers: pancreatic [(Cheng, J. Q. et al., Proc. Natl. Acad. Sci., 93, pp. 3636-3641, 1996), Neoplasia, 3, pp. 278, 2001)], lung [(Brognard, J. et al., Cancer Res., 61, pp. 3986-3997, 2001), Neoplasia, 3, pp. 278, 2001)], ovarian [(Hayakawa, J. et al., Cancer Res., 60, pp. 5988-5994, 2000), Neoplasia, 3, pp. 278, 2001)], breast (Mol. Cancer Ther., 1, pp. 707, 2002), colon [(Neoplasia, 3, pp. 278, 2001), (Arico, S. et al., J. Biol. Chem., 277, pp. 27613-27621, 2002)], cervical (Neoplasia, 3, pp. 278, 2001), prostate [(Endocrinology, 142, pp. 4795, 2001), (Thakkar, H. et al. J. Biol. Chem., 276, pp. 38361-38369, 2001), (Chen, X. et al., Oncogene, 20, pp. 6073-6083, 2001)] and brain (glioblastomas) [(Flynn, P. et al., Curr. Biol., 10, pp. 1439-1442, 2000)].

One kinase family of interest is Rho-associated coiled-coil forming protein serine/threonine kinase (ROCK), which is believed to be an effector of Ras-related small GTPase Rho. The ROCK family includes p160ROCK (ROCK-1) (Ishizaki et al., EMBO J. 1996, 15, 1885-1893) and ROKa/Rho-kinase/ROCK-II (Leung et al., J. Biol. Chem. 1995, 270, 29051-29054; Matsui et al., EMBO J. 1996, 15, 2208-2216; Nakagawa et al., FEBS Lett. 1996, 392, 189-193), protein kinase PKN (Amano et al., Science 1996, 271, 648-650; Watanabe et al., Science 1996, 271, 645-648), and citron and citron kinase (Madaule et al. Nature, 1998, 394, 491-494; Madaule et al., FEBS Lett. 1995, 377, 243-248). The ROCK family of kinases have been shown to be involved in a variety of functions including Rho-induced formation of actin stress fibers and focal adhesions (Leung et al., Mol. Cell Biol. 1996, 16, 5313-5327; Amano et al., Science, 1997, 275, 1308-1311; Ishizaki et al., FEBS Lett. 1997, 404, 118-124) and in downregulation of myosin phosphatase (Kimura et al., Science, 1996, 273, 245-248), platelet activation (Klages et al., J. Cell. Biol., 1999, 144, 745-754), aortic smooth muscle contraction by various stimuli (Fu et al., FEBS Lett., 1998, 440, 183-187), thrombin-induced responses of aortic smooth muscle cells (Seasholtz et al., Cir. Res., 1999, 84, 1186-1193), hypertrophy of cardiomyocytes (Kuwahara et al., FEBS Lett., 1999, 452, 314-318), bronchial smooth muscle contraction (Yoshii et al., Am. J. Respir. Cell Mol. Biol., 1999, 20, 1190-1200), smooth muscle contraction and cytoskeletal reorganization of non-muscle cells (Fukata et al., Trends in Pharm. Sci 2001, 22, 32-39), activation of volume-regulated anion channels (Nilius et al., J. Physiol., 1999, 516, 67-74), neurite retraction (Hirose et al., J. Cell. Biol., 1998, 141, 1625-1636), neutrophil chemotaxis (Niggli, FEBS Lett., 1999, 445, 69-72), wound healing (Nobes and Hall, J. Cell. Biol., 1999, 144, 1235-1244), tumor invasion (Itoh et al., Nat. Med., 1999, 5, 221-225) and cell transformation (Sahai et al., Curr. Biol., 1999, 9, 136-145). More specifically, ROCK has been implicated in various diseases and disorders including hypertension (Satoh et al., J. Clin. Invest. 1994, 94, 1397-1403; Mukai et al., FASEB J. 2001, 15, 1062-1064; Uehata et al., Nature 1997, 389, 990-994; Masumoto et al., Hypertension, 2001, 38, 1307-1310), cerebral vasospasm (Sato et al., Circ. Res. 2000, 87, 195-200; Miyagi et al., J. Neurosurg. 2000, 93, 471-476; Tachibana et al., Acta Neurochir (Wien) 1999, 141, 13-19), coronary vasospasm (Shimokawa et al., Jpn. Cir. J. 2000, 64, 1-12; Kandabashi et al., Circulation 2000, 101, 1319-1323; Katsumata et al., Circulation 1997, 96, 4357-4363; Shimokawa et al., Cardiovasc. Res. 2001, 51, 169-177; Utsunomiya et al., J. Pharmacol. 2001, 134, 1724-1730; Masumoto et al., Circulation 2002, 105, 1545-1547), bronchial asthma (Chiba et al., Comp. Biochem. Physiol. C Pharmacol. Toxicol. Endocrinol. 1995, 11, 351-357; Chiba et al., Br. J. Pharmacol. 1999, 127, 597-600; Chiba et al., Br. J. Pharmacol. 2001, 133, 886-890; Iizuka et al., Eur. J. Pharmacol. 2000, 406, 273-279), preterm labor (Niro et al., Biochem. Biophys. Res. Commun. 1997, 230, 356-359; Tahara et al., Endocrinology 2002, 143, 920-929; Kupittayanant et al., Pflugers Arch. 2001, 443, 112-114), erectile dysfunction (Chitaley et al., Nat. Med. 2001, 7, 119-122; Mills et al., J. Appl. Physiol. 2001, 91, 1269-1273), glaucoma (Honjo et al., Arch. Ophthalmol. 2001, 1171-1178; Rao et al., Invest. Ophthalmol. Vis. Sci. 2001, 42, 1029-1037), vascular smooth muscle cell proliferation (Shimokawa et al., Cardiovasc. Res. 2001, 51, 169-177; Morishige et al., Arterioscler. Thromb. Vasc. Biol. 2001, 21, 548-554; Eto et al., Am. J. Physiol. Heart Circ. Physiol. 2000, 278, H1744-H1750; Sawada et al., Circulation 2000, 101, 2030-2023; Shibata et al., Circulation 2001, 103, 284-289), myocardial hypertrophy (Hoshijima et al., J. Biol. Chem. 1998, 273, 7725-77230; Sah et al., J. Biol. Chem. 1996, 271, 31185-31190; Kuwahara et al., FEBS Lett. 1999, 452, 314-318; Yanazume et al., J. Biol. Chem. 2002, 277, 8618-8625), malignoma (Itoh et al., Nat. Med. 1999, 5, 221-225; Genda et al., Hepatology 1999, 30, 1027-1036; Somlyo et al., Biochem. Biophys. Res. Commun. 2000, 269, 652-659), ischemia/reperfusion-induced injury (Ikeda et al., J. of Surgical Res. 2003, 109, 155-160; Miznuma et al. Transplantation 2003, 75, 579-586), endothelial dysfunction (Hernandez-Perera et al., Circ. Res. 2000, 87, 616-622; Laufs et al., J. Biol. Chem. 1998, 273, 24266-24271; Eto et al., Circ. Res. 2001, 89, 583-590), Crohn's Disease and colitis (Segain et al. Gastroenterology 2003, 124(5), 1180-1187), neurite outgrowth (Fournier et al. J. Neurosci. 2003, 23, 1416-1423), Raynaud's Disease (Shimokawa et al. J. Cardiovasc. Pharmacol. 2002, 39, 319-327), angina (Utsunomiya et al. Br. J. Pharmacol. 2001, 134, 1724-1730; Masumoto et al, Circulation 2002, 105, 1545-1547; Shimokawa et al, J. Cardiovasc. Pharmacol., 2002, 40, 751-761; Satoh et al., Jpn. J. Pharmacol., 2001, 87, 34-40), Alzheimer's disease (Zhou et al., Science 2003, 302, 1215-1218), benign prostatic hyperplasia (Rees et al., J. Urology, 2003, 170, 2517-2522), and atherosclerosis (Retzer et al. FEBS Lett. 2000, 466, 70-74; Ishibashi et al. Biochim. Biophys. Acta 2002, 1590, 123-130). Accordingly, the development of inhibitors of ROCK kinase would be useful as therapeutic agents for the treatment of disorders implicated in the ROCK kinase pathway.

Accordingly, there is a great need to develop inhibitors of AKT, PDK1, p70S6K, and ROCK protein kinases that are useful in treating various diseases or conditions associated with AKT, PDK1, p70S6K, and ROCK activation, particularly given the inadequate treatments currently available for the majority of these disorders.

### SUMMARY OF THE INVENTION

It has now been found that compounds of this invention, and pharmaceutically acceptable compositions thereof, are effective as inhibitors of AKT, PDK1, p70S6K, and ROCK protein kinases. These compounds have the formula I: 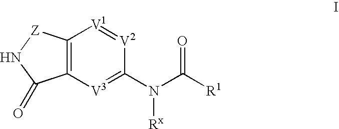
or a pharmaceutically acceptable salt thereof, wherein Z, V¹, V², V³, R^{x}, and R¹ are as defined below.

These compounds, and pharmaceutically acceptable compositions thereof, are useful for treating or lessening the severity of a variety of disorders, including allergic disorders such as asthma, inflammatory disease, proliferative disorders, and neurological disorders.

### DESCRIPTION OF THE INVENTION

The present invention relates to a compound of formula I: 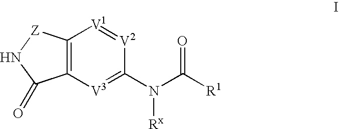
or a pharmaceutically acceptable salt thereof, wherein:
Z is —CH₂— or —C(O)—;
R¹ is selected from T-R, T-Ar, or T-C(R)(T-Ar)R², wherein:

R and R² optionally form a 5-7 membered saturated or partially unsaturated ring having 0-4 heteroatoms independently selected from nitrogen, oxygen, or sulfur;
R^{x} is T-R or T-Ar, or:

R^{x} and R¹ are taken together to form an optionally substituted 5-7 membered saturated or partially unsaturated ring having 1-4 heteroatoms independently selected from nitrogen, oxygen, or sulfur;
each T is independently selected from a valence bond or a C₁₋₆ alkylidene chain, wherein up to two methylene units of T are optionally, and independently, replaced by —O—, —N(R)—, —S—, —S(O)—, —N(R)C(O)—, —C(O)N(R)—, —C(O)—, or —SO₂—;
each R is independently selected from hydrogen or an optionally substituted C₁₋₆ aliphatic group, or:

two R groups on the same nitrogen, taken together with the nitrogen atom attached thereto, form a 5-7 membered saturated, partially unsaturated, or fully unsaturated ring having 1-3 heteroatoms independently selected from nitrogen, oxygen, or sulfur;
each Q is independently selected from a valence bond or a C₁₋₄ alkylidene chain;
each Ar is independently an optionally substituted ring selected from a 5-7 membered saturated, partially unsaturated, or fully unsaturated monocyclic ring having 0-4 heteroatoms independently selected from nitrogen, oxygen, or sulfur, or an 8-10 membered saturated, partially unsaturated, or fully unsaturated bicyclic ring having 0-4 heteroatoms independently selected from nitrogen, oxygen, or sulfur;
R² is selected from R′, Ar¹, Q-OR³, Q-OC(O)R³, Q-CONHR⁴, Q-OC(O)NHR⁴, Q-SR³, Q-N(R⁴)₂, N(R)(Q-Ar), N(R)C(O)Q-N(R⁴)₂, or N(R)Q-N(R⁴)₂;
R′ is an optionally substituted C₁₋₆ aliphatic group;
each R³ is independently selected from R or Ar;
each R⁴ is independently selected from R, COR³, CO₂R³, CON(R³)₂, SO₂R³, SO₂N(R³)₂, or Ar¹;
V¹, V² and V³ are each independently selected from nitrogen or C(R⁵);
each R⁵ is independently selected from R, Ar¹, halogen, CN, NO₂, OR, SR, N(R⁴)₂, N(R)COR, N(R)CON(R⁴)₂, N(R)C(O)OR, CON(R⁴)₂, OC(O)N(R⁴)₂, CO₂R, OC(O)R, N(R)SO₂R, N(R)SO₂N(R⁴)₂, SO₂R, or SO₂N(R⁴)₂; and
each Ar¹ is independently selected from an optionally substituted 5-7 membered saturated, partially unsaturated, or fully unsaturated monocyclic ring having 0-4 heteroatoms independently selected from nitrogen, oxygen, or sulfur.

As used herein, the following definitions shall apply unless otherwise indicated. The phrase “optionally substituted” is used interchangeably with the phrase “substituted or unsubstituted.” Unless otherwise indicated, an optionally substituted group may have a substituent at each substitutable position of the group, and each substitution is independent of the other.

The term “aliphatic” or “aliphatic group” as used herein means a straight-chain or branched C₁-C₁₂ hydrocarbon chain that is completely saturated or that contains one or more units of unsaturation, or a monocyclic C₃-C₈ hydrocarbon or bicyclic C₈-C₁₂ hydrocarbon that is completely saturated or that contains one or more units of unsaturation, but which is not aromatic (also referred to herein as “carbocycle” or “cycloalkyl”), that has a single point of attachment to the rest of the molecule wherein any individual ring in said bicyclic ring system has 3-7 members. For example, suitable aliphatic groups include, but are not limited to, linear or branched or alkyl, alkenyl, alkynyl groups and hybrids thereof such as (cycloalkyl)alkyl, (cycloalkenyl)alkyl or (cycloalkyl)alkenyl.

The terms “alkyl”, “alkoxy”, “hydroxyalkyl”, “alkoxyalkyl”, and “alkoxycarbonyl”, used alone or as part of a larger moiety includes both straight and branched chains containing one to twelve carbon atoms. The terms “alkenyl” and “alkynyl” used alone or as part of a larger moiety shall include both straight and branched chains containing two to twelve carbon atoms.

The terms “haloalkyl”, “haloalkenyl” and “haloalkoxy” means alkyl, alkenyl or alkoxy, as the case may be, substituted with one or more halogen atoms. The term “halogen” means F, Cl, Br, or I.

The term “heteroatom” means nitrogen, oxygen, or sulfur and includes any oxidized form of nitrogen and sulfur, and the quaternized form of any basic nitrogen. Also the term “nitrogen” includes a substitutable nitrogen of a heterocyclic ring. As an example, in a saturated or partially unsaturated ring having 0-4 heteroatoms selected from oxygen, sulfur or nitrogen, the nitrogen may be N (as in 3,4-dihydro-2H-pyrrolyl), NH (as in pyrrolidinyl) or NR⁺ (as in N-substituted pyrrolidinyl).

The term “aryl” used alone or as part of a larger moiety as in “aralkyl”, “aralkoxy”, or “aryloxyalkyl”, refers to monocyclic, bicyclic and tricyclic ring systems having a total of five to fourteen ring members, wherein at least one ring in the system is aromatic and wherein each ring in the system contains 3 to 7 ring members. The term “aryl” may be used interchangeably with the term “aryl ring”.

The term “heterocycle”, “heterocyclyl”, or “heterocyclic” as used herein means non-aromatic, monocyclic, bicyclic or tricyclic ring systems having five to fourteen ring members in which one or more ring members is a heteroatom, wherein each ring in the system contains 3 to 7 ring members.

The term “heteroaryl”, used alone or as part of a larger moiety as in “heteroaralkyl” or “heteroarylalkoxy”, refers to monocyclic, bicyclic and tricyclic ring systems having a total of five to fourteen ring members, wherein at least one ring in the system is aromatic, at least one ring in the system contains one or more heteroatoms, and wherein each ring in the system contains 3 to 7 ring members. The term “heteroaryl” may be used interchangeably with the term “heteroaryl ring” or the term “heteroaromatic”.

An aryl (including aralkyl, aralkoxy, aryloxyalkyl and the like) or heteroaryl (including heteroaralkyl and heteroarylalkoxy and the like) group may contain one or more substituents. Suitable substituents on the unsaturated carbon atom of an aryl, heteroaryl, aralkyl, or heteroaralkyl group are selected from halogen, oxo, N₃, —R°, —OR°, —SR°, 1,2-methylene-dioxy, 1,2-ethylenedioxy, protected OH (such as acyloxy), phenyl (Ph), Ph substituted with R°, —O(Ph), O-(Ph) substituted with R°, —CH₂(Ph), —CH₂(Ph) substituted with R°, —CH₂CH₂(Ph), —CH₂CH₂(Ph) substituted with R°, —NO₂, —CN, —N(R°)₂, —NR°C(O)R°, —NR°C(O)N(R°)₂, —NR°CO₂R°, —NR°NR°C(O)R°, —NR°NR°C(O)N(R°)₂, —NR°NR°CO₂R°, —C(O)C(O)R°, —C(O)CH₂C(O)R°, —CO₂R°, —C(O)R°, —C(O)N(R°)₂, —OC(O)N(R°)₂, —S(O)₂R°, —SO₂N(R°)₂, —S(O)R°, —NR°SO₂N(R°)₂, —NR°SO₂R°, —C(═S)N(R°)₂, —C(═NH)—N(R°)₂, or —(CH₂)_{y}NHC(O)R°, wherein y is 0-4, each R° is independently selected from hydrogen, optionally substituted C₁₋₆ aliphatic, an unsubstituted 5-6 membered heteroaryl or heterocyclic ring having 0-4 heteroatoms independently selected from nitrogen, oxygen, or sulfur, phenyl (Ph), —O(Ph), or —CH₂(Ph)-CH₂(Ph). Substituents on the aliphatic group of R° are selected from NH₂, NH(C₁₋₄ aliphatic), N(C₁₋₄ aliphatic)₂, halogen, C₁₋₄ aliphatic, OH, O—(C₁₋₄ aliphatic), NO₂, CN, CO₂H, CO₂(C₁₋₄ aliphatic), —O(halo C₁₋₄ aliphatic), or halo C₁₋₄ aliphatic.

An aliphatic group or a non-aromatic heterocyclic ring may contain one or more substituents. Suitable substituents on the saturated carbon of an aliphatic group or of a non-aromatic heterocyclic ring are selected from those listed above for the unsaturated carbon of an aryl or heteroaryl group and the following: ═O, ═S, ═NNHR*, ═NN(R*)₂, ═N—, ═NNHC(O)R*, ═NNHCO₂(alkyl), ═NNHSO₂(alkyl), or ═NR*, where each R* is independently selected from hydrogen or an optionally substituted C₁₋₆ aliphatic. Substituents on the aliphatic group of R* are selected from NH₂, NH(C₁₋₄ aliphatic), N(C₁₋₄ aliphatic)₂, halogen, C₁₋₄ aliphatic, OH, O—(C₁₋₄ aliphatic), NO₂, CN, CO₂H, CO₂(C₁₋₄ aliphatic), —O(halo C₁₋₄ aliphatic), or halo C₁₋₄ aliphatic.

Substituents on the nitrogen of a non-aromatic heterocyclic ring are selected from —R⁺, —N(R⁺)₂, —C(O)R⁺, —CO₂R⁺, —C(O)C(O)R⁺, —C(O)CH₂C(O)R⁺, —SO₂R⁺, —SO₂N(R⁺)₂, —C(═S)N(R⁺)₂, —C(═NH)—N(R⁺)₂, or —NR⁺SO₂R⁺; wherein R⁺ is hydrogen, an optionally substituted C₁₋₆ aliphatic, optionally substituted phenyl (Ph), optionally substituted —O(Ph), optionally substituted —CH₂(Ph), optionally substituted —CH₂CH₂(Ph), or an unsubstituted 5-6 membered heteroaryl or heterocyclic ring. Substituents on the aliphatic group or the phenyl ring of R⁺ are selected from NH₂, NH(C₁₋₄ aliphatic), N(C₁₋₄ aliphatic)₂, halogen, C₁₋₄ aliphatic, OH, O—(C₁₋₄ aliphatic), NO₂, CN, CO₂H, CO₂(C₁₋₄ aliphatic), —O(halo C₁₋₄ aliphatic), or halo C₁₋₄ aliphatic.

The term “alkylidene chain” refers to a straight or branched carbon chain that may be fully saturated or have one or more units of unsaturation and has two points of connection to the rest of the molecule.

The compounds of this invention are limited to those that are chemically feasible and stable. Therefore, a combination of substituents or variables in the compounds described above is permissible only if such a combination results in a stable or chemically feasible compound. A stable compound or chemically feasible compound is one in which the chemical structure is not substantially altered when kept at a temperature of 40° C. or less, in the absence of moisture or other chemically reactive conditions, for at least a week.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention. Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by a ¹³C- or ¹⁴C-enriched carbon are within the scope of this invention.

Compounds of this invention may exist in alternative tautomeric forms. Unless otherwise indicated, the representation of either tautomer is meant to include the other.

According to another embodiment, the present invention relates to a compound of formula I: 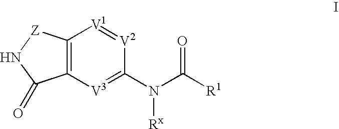
or a pharmaceutically acceptable salt thereof, wherein:
Z is —CH₂— or —C(O)—;
R¹ is selected from T-R, T-Ar, or T-C(R)(T-Ar)R², wherein:

R and R² optionally form a 5-7 membered saturated or partially unsaturated ring having 0-4 heteroatoms independently selected from nitrogen, oxygen, or sulfur;
R^{x} is T-R or T-Ar, or:

R^{x} and R¹ are taken together to form an optionally substituted 5-7 membered saturated or partially unsaturated ring having 1-4 heteroatoms independently selected from nitrogen, oxygen, or sulfur;
each T is independently selected from a valence bond or a C₁₋₆ alkylidene chain, wherein up to two methylene units of T are optionally, and independently, replaced by —O—, —N(R)—, —S—, —N(R)C(O)—, —C(O)N(R)—, —C(O)—, or —SO₂—;
each R is independently selected from hydrogen or an optionally substituted C₁₋₆ aliphatic group, or:

two R groups on the same nitrogen, taken together with the nitrogen atom attached thereto, form a 5-7 membered saturated, partially unsaturated, or fully unsaturated ring having 1-3 heteroatoms independently selected from nitrogen, oxygen, or sulfur;
each Q is independently selected from a valence bond or a C₁₋₄ alkylidene chain;
each Ar is independently an optionally substituted ring selected from a 5-7 membered saturated, partially unsaturated, or fully unsaturated monocyclic ring having 0-4 heteroatoms independently selected from nitrogen, oxygen, or sulfur, or an 8-10 membered saturated, partially unsaturated, or fully unsaturated bicyclic ring having 0-4 heteroatoms independently selected from nitrogen, oxygen, or sulfur;
R² is selected from R′, Ar¹, Q-OR³, Q-OC(O)R³, Q-CONHR⁴, Q-OC(O)NHR⁴, Q-SR³, Q-N(R⁴)₂, N(R)(Q-Ar), N(R)C(O)Q-N(R⁴)₂, or N(R)Q-N(R⁴)₂;
R′ is an optionally substituted C₁₋₆ aliphatic group;
each R³ is independently selected from R or Ar;
each R⁴ is independently selected from R, COR³, CO₂R³, CON(R³)₂, SO₂R³, SO₂N(R³)₂, or Ar¹;
V¹, V² and V³ are each independently selected from nitrogen or C(R⁵);
each R⁵ is independently selected from R, Ar¹, halogen, CN, NO₂, OR, SR, N(R⁴)₂, N(R)COR, N(R)CON(R⁴)₂, N(R)C(O)OR, CON(R⁴)₂, OC(O)N(R⁴)₂, CO₂R, OC(O)R, N(R)SO₂R, N(R)SO₂N(R⁴)₂, SO₂R, or SO₂N(R⁴)₂; and
each Ar¹ is independently selected from an optionally substituted 5-7 membered saturated, partially unsaturated, or fully unsaturated monocyclic ring having 0-4 heteroatoms independently selected from nitrogen, oxygen, or sulfur;
provided that said compound is other than the group consisting of:

N-(2,3-dihydro-3-oxo-1H-isoindol-5-yl)-benzamide,

5-(3,4-dimethylphenoxy)-[2,5′-bi-2H-isoindole]-1,1′,3,3′-tetrone,

N-(2,3-dihydro-1,3-dioxo-1H-isoindol-5-yl)-2-methoxy-benzamide,

N-(2,3-dihydro-1,3-dioxo-1H-isoindol-5-yl)-2-ethoxy-benzamide,

N-(2,3-dihydro-1,3-dioxo-1H-isoindol-5-yl)-2-(3,4-dimethylphenoxy)-acetamide,

N-(2,3-dihydro-1,3-dioxo-1H-isoindol-5-yl)-2-(2,5-dimethylphenoxy)-acetamide,

N-(2,3-dihydro-1,3-dioxo-1H-isoindol-5-yl)-2-(3-methoxyphenoxy)-acetamide,

N-(2,3-dihydro-1,3-dioxo-1H-isoindol-5-yl)-1,3-dihydro-5-nitro-1,3-dioxo-(2H-isoindole-2-acetamide),

N-(2,3-dihydro-1,3-dioxo-1H-isoindol-5-yl)-1,3-dihydro-1,3-dioxo-(2H-isoindole-2-acetamide),

N-(2,3-dihydro-1,3-dioxo-1H-isoindol-5-yl)-2-[4-(1-methylpropyl)phenoxy]-acetamide,

N-(2,3-dihydro-1,3-dioxo-1H-isoindol-5-yl)-2-(4-iodophenoxy)-acetamide,

N-(2,3-dihydro-1,3-dioxo-1H-isoindol-5-yl)-2-(phenylthio)-acetamide,

N-(2,3-dihydro-1,3-dioxo-1H-isoindol-5-yl)-3-(3-nitrophenyl)-2-propenamide,

N-(2,3-dihydro-1,3-dioxo-1H-isoindol-5-yl)-3-phenyl-2-propenamide,

3-(5-bromo-2-methoxyphenyl)-N-(2,3-dihydro-1,3-dioxo-1H-isoindol-5-yl)-2-propenamide,

12-(2,3-dihydro-1,3-dioxo-1H-isoindol-5-yl)-11H-benzo[5,6][1,4]benzodioxocino[2,3-f]isoindole-11,13(12H)-dione,

N-(2,3-dihydro-1,3-dioxo-1H-isoindol-5-yl)-2-phenoxy-propanamide,

2-[(4-chlorophenyl)thio]-N-(2,3-dihydro-1,3-dioxo-1H-isoindol-5-yl)-acetamide,

N-[3-[[(2,3-dihydro-1,3-dioxo-1H-isoindol-5-yl)amino]carbonyl]phenyl]-2-furancarboxamide,

2-(4-bromophenoxy)-N-(2,3-dihydro-1,3-dioxo-1H-isoindol-5-yl)-acetamide,

2-bromo-N-(2,3-dihydro-1,3-dioxo-1H-isoindol-5-yl)-benzamide,

N-(2,3-dihydro-1,3-dioxo-1H-isoindol-5-yl)-2-[4-(1-methylethyl)phenoxy]-acetamide,

2-(2-chlorophenoxy)-N-(2,3-dihydro-1,3-dioxo-1H-isoindol-5-yl)-acetamide,

4-chloro-N-(2,3-dihydro-1,3-dioxo-1H-isoindol-5-yl)-benzeneacetamide,

4-[[bis(2-cyanoethyl)amino]sulfonyl]-N-(2,3-dihydro-1,3-dioxo-1H-isoindol-5-yl)-benzamide,

N-(2,3-dihydro-1,3-dioxo-1H-isoindol-5-yl)-2,3,3,3-tetrafluoro-2-methoxy-propanamide,

N-(2,3-dihydro-1,3-dioxo-1H-isoindol-5-yl)-2,2,2-trifluoro-acetamide,

N-(2,3-dihydro-1,3-dioxo-1H-isoindol-5-yl)-benzeneacetamide,

N-(2,3-dihydro-1,3-dioxo-1H-isoindol-5-yl)-2-furancarboxamide,

N-(2,3-dihydro-1,3-dioxo-1H-isoindol-5-yl)-1,2,3,6-tetrahydro-2,6-dioxo-4-pyrimidinecarboxamide,

N-(2,3-dihydro-1,3-dioxo-1H-isoindol-5-yl)-2-phenoxy-acetamide,

1-(4-chlorophenyl)-N-(2,3-dihydro-1,3-dioxo-1H-isoindol-5-yl)-1,2,5,6,7,8-hexahydro-7,7-dimethyl-2,5-dioxo-3-quinolinecarboxamide,

N-(2,3-dihydro-1,3-dioxo-1H-isoindol-5-yl)-benzenepropanamide,

N-(2,3-dihydro-1,3-dioxo-1H-isoindol-5-yl)-1,2,5,6,7,8-hexahydro-7,7-dimethyl-2,5-dioxo-1-phenyl-3-quinolinecarboxamide,

2-[[(2,4-dichlorophenyl)methyl]thio]-N-(2,3-dihydro-1,3-dioxo-1H-isoindol-5-yl)-acetamide,

4-amino-N-(2,3-dihydro-1,3-dioxo-1H-isoindol-5-yl)-1-methyl-1H-pyrazole-3-carboxamide,

N-(2,3-dihydro-1,3-dioxo-1H-isoindol-5-yl)-1-methyl-4-nitro-1H-pyrazole-3-carboxamide,

4-(acetyloxy)-N-(2,3-dihydro-1,3-dioxo-1H-isoindol-5-yl)-benzamide,

3-chloro—N-(2,3-dihydro-1,3-dioxo-1H-isoindol-5-yl)-benzo[b]thiophene-2-carboxamide,

N-(2,3-dihydro-1,3-dioxo-1H-isoindol-5-yl)-2-thiophenecarboxamide,

N-(2,3-dihydro-1,3-dioxo-1H-isoindol-5-yl)-2-methyl-propanamide,

4-amino-N-(2,3-dihydro-1,3-dioxo-1H-isoindol-5-yl)-benzamide,

3,5-diamino-N-(2,3-dihydro-1,3-dioxo-1H-isoindol-5-yl)-benzamide,

N-(2,3-dihydro-1,3-dioxo-1H-isoindol-5-yl)-cyclohexanecarboxamide,

2,4-dichloro-N-(2,3-dihydro-1,3-dioxo-1H-isoindol-5-yl)-benzamide,

4-[[(2,3-dihydro-1,3-dioxo-1H-isoindol-5-yl)amino]carbonyl]-benzoic acid, methyl ester

N-(2,3-dihydro-1,3-dioxo-1H-isoindol-5-yl)-4-nitro-benzamide,

N-(2,3-dihydro-1,3-dioxo-1H-isoindol-5-yl)-3,5-dinitro-benzamide,

4-[(diethylamino)sulfonyl]-N-(2,3-dihydro-1,3-dioxo-1H-isoindol-5-yl)-benzamide,

N-(2,3-dihydro-1,3-dioxo-1H-isoindol-5-yl)-1-[(4-methylphenyl)sulfonyl]-cyclopropanecarboxamide,

4-chloro-1H-isoindole-1,3(2H)-dione, 2-(2,3-dihydro-1,3-dioxo-1H-isoindol-5-yl)-4-nitro-[2,5′-Bi-2H-isoindole]-1,1′,3,3′-tetrone,

N-(2,3-dihydro-1,3-dioxo-1H-isoindol-5-yl)-2-methyl-2-propenamide,

N-(2,3-dihydro-1,3-dioxo-1H-isoindol-5-yl)-3-hydroxy-2-naphthalenecarboxamide,

N-(2,3-dihydro-1,3-dioxo-1H-isoindol-5-yl)-4-methoxy-benzamide,

4-chloro-N-(2,3-dihydro-1,3-dioxo-1H-isoindol-5-yl)-benzamide,

N-(2,3-dihydro-1,3-dioxo-1H-isoindol-5-yl)-benzamide,

2-chloro-N-(2,3-dihydro-1,3-dioxo-1H-isoindol-5-yl)-acetamide, and

N-(2,3-dihydro-1,3-dioxo-1H-isoindol-5-yl)-acetamide.

One embodiment of the present invention relates to a compound of formula Ia: 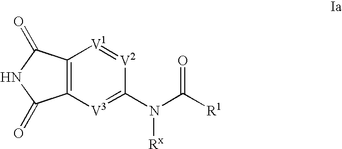
or a pharmaceutically acceptable salt thereof, wherein V¹, V², V³, and R¹ are as defined above for compounds of formula I.

One aspect of the present invention relates to a compound of formula I or Ia wherein R^{x} is hydrogen.

Another aspect of the present invention relates to a compound of formula I or Ia wherein R^{x} is T-Ar.

Yet another aspect of the present invention relates to a compound of formula I or Ia wherein R^{x} and R¹ are taken together to form an optionally substituted 5-7 membered saturated or partially unsaturated ring having 1-4 heteroatoms independently selected from nitrogen, oxygen, or sulfur.

According to one embodiment, the ring formed by the R^{x} and R¹ groups of formula I or Ia is a 5-6 membered saturated ring having 1-2 heteroatoms independently selected from nitrogen, oxygen, or sulfur.

According to another embodiment, the present invention relates to a compound of formula I or Ia wherein V¹ is N, V² is C(R⁵), and V³ is C(R⁵).

Other compounds of formula I or Ia include those wherein V¹ is C(R⁵), V² is C(R⁵), and V³ is C(R⁵) or N.

Another embodiment of the present invention relates to a compound of formula I or Ia wherein V¹ is C(R⁵), V² is C(R⁵) or N, and V³ is N.

Another embodiment of the present invention relates to a compound of formula I or Ia wherein V¹ is CH, V² is CH, and V³ is CH.

According to another embodiment, the present invention relates to a compound of formula Ia wherein V¹ is CH, and V² and V³ are each N.

When R¹ of formula I or Ia is T-Ar, such Ar groups include an optionally substituted ring selected from a 5-6 membered saturated, partially unsaturated, or fully unsaturated monocyclic ring having 0-4 heteroatoms independently selected from nitrogen, oxygen, or sulfur, or a 9-10 membered saturated, partially unsaturated, or fully unsaturated bicyclic ring having 0-4 heteroatoms independently selected from nitrogen, oxygen, or sulfur. Examples of such monocyclic rings include phenyl, pyridyl, pyrimidinyl, pyridonyl, furanyl, tetrazolyl, thienyl, cyclopentyl, cyclohexyl, and cycloheptyl. Examples of such bicyclic rings include benzo[1,3]dioxolyl, indan-1-onyl, naphthyl, benzothiophenyl, 2,3-dihydro-1H-isoindolyl, indanyl, benzofuranyl, and indolyl.

When present, substituents on the Ar ring of R¹ include R°, halogen, oxo, OR°, phenyl, optionally substituted dialkylamino, haloalkyl, C(O)R°, NHC(O)R, or SR°. Examples of such substituents include chloro, bromo, fluoro, OH, OMe, NHC(O)CH₃, OEt, C(O)phenyl, Ophenyl, N(CH₂CH₂Cl)₂, N(Me)₂, CF₃, and SCF₃. Other examples of Ar groups of formula I or Ia also include those shown in Table 1 below.

When the R¹ group of formula I or Ia is T-C(R)(T-Ar)R², R² groups include R′, Q-OR³, Q-N(R⁴)₂, Ar¹, N(R)C(O)Q-N(R⁴)₂, and N(R)Q-N(R⁴)₂. Examples of such R² groups include CH₂OH, OH, NH₂, CH₂NH₂, CH₂NHMe, CH₂N(Me)₂, CH₂CH₂NH₂, CH₂CH₂NHMe, CH₂CH₂N(Me)₂, CH₂C(Me)₂NH₂, CH₂C(Me)₂CHMe, NHCO₂t-butyl, phenyl, cyclopentyl, methyl, ethyl, isopropyl, cyclopropyl, NH(CH₂)₃NH₂, NH(CH₂)₂NH₂, NH(CH₂)₂NHEt, NHCH₂pyridyl, NHSO₂phenyl, NHC(O)CH₂C(O)Ot-butyl, NHC(O)CH₂NH₃, and NHCH₂-imidazol-4-yl.

According to another embodiment, the R² group of formula I or Ia is selected from OH, NH₂, CH₂NH₂, CH₂NHMe, CH₂N(Me)₂, CH₂CH₂NH₂, CH₂CH₂NHMe, CH₂CH₂N(Me)₂, CH₂C(Me)₂NH₂, CH₂C(Me)₂CHMe, NHCO₂t-butyl, phenyl, NH(CH₂)₃NH₂, NH(CH₂)₂NH₂, NH(CH₂)₂NHEt, NHCH₂pyridyl, NHSO₂phenyl, NHC(O)CH₂C(O)Ot-butyl, NHC(O)CH₂NH₃, and NHCH₂-imidazol-4-yl.

According to another embodiment, the R² group of formula I or Ia is selected from CH₂CH₂NH₂.

When present, rings formed by the R and R² moieties of the T-C(R)(T-Ar)R² group of R¹ are selected from a 5-6 membered saturated ring having 0-2 heteroatoms independently selected from nitrogen, oxygen, or sulfur. Examples of such rings formed by R and R² include piperidinyl, pyrrolidinyl, piperazinyl, morpholinyl, and thiomorpholinyl.

When the R¹ group of formula I or Ia is T-C(R)(T-Ar)R², Ar groups of the T-C(R)(T-Ar)R² moiety are selected from an optionally substituted 5-6 membered saturated, partially unsaturated, or fully unsaturated monocyclic ring having 0-4 heteroatoms independently selected from nitrogen, oxygen, or sulfur, or an optionally substituted 9-10 membered saturated, partially unsaturated, or fully unsaturated bicyclic ring having 0-4 heteroatoms independently selected from nitrogen, oxygen, or sulfur. Examples of such monocyclic rings include phenyl, pyridyl, furanyl, pyridone, and thienyl. Examples of such bicyclic rings include benzo[1,3]dioxolyl, naphthyl, indanyl, and indolyl. When present, substituents on the Ar ring of the T-C(R)(T-Ar)R² group of R¹ include R°, halogen, OR°, phenyl, N(R°)₂, NHC(O)R°, or SR°. Examples of such groups include fluoro, chloro, bromo, CF₃, OH, OMe, OPh, OCH₂Ph, SMe, NH₂, NHC(O)Me, methyl, ethyl, isopropyl, isobutyl, and cyclopropyl.

R⁵ groups of formula I or Ia, when present, are selected from halogen, R, and Ar¹. Other R⁵ groups of formula I or Ia, when present, are selected from halogen, optionally substituted C₁₋₄ aliphatic, or an optionally substituted 5-6 membered saturated, partially unsaturated, or fully unsaturated monocyclic ring having 0-4 heteroatoms independently selected from nitrogen, oxygen, or sulfur. Examples of such groups include chloro, bromo, methyl, ethyl, t-butyl, cyclopropyl, isopropyl, phenyl, and pyridyl.

According to another embodiment, for compounds of formula I or Ia, Z is —C(O)—, V¹, V², and V³ are C(R⁵) wherein R⁵ is hydrogen, R^{x} is hydrogen, and R¹ is T-Ar.

According to another embodiment, for compounds of formula I or Ia, Z is —C(O)—, V¹, V², and V³ are C(R⁵) wherein R⁵ is hydrogen, R^{x} is hydrogen, R¹ is T-Ar, and T is selected from —CH₂—.

In another embodiment, for compounds of formula I or Ia, Z is —C(O)—, V¹, V², and V³ are C(R⁵) wherein R⁵ is hydrogen, R^{x} is hydrogen, R¹ is T-Ar, T is selected from —CH₂—, and Ar is a 5-6 membered optionally substituted, fully unsaturated ring having 0-2 heteroatoms independently selected from nitrogen.

In yet another embodiment, for compounds of formula I or Ia, Z is —C(O)—, V¹, V², and V³ are C(R⁵) wherein R⁵ is hydrogen, R^{x} is hydrogen, R¹ is T-Ar, T is selected from —CH₂—, and Ar is a 6 membered optionally substituted, fully unsaturated ring having 0-1 heteroatoms independently selected from nitrogen.

In another embodiment, for compounds of formula I or Ia, Z is —C(O)—, V¹, V², and V³ are C(R⁵) wherein R⁵ is hydrogen, R^{x} is hydrogen, R¹ is T-Ar, T is selected from —CH₂—, and Ar is a 6 membered optionally substituted, fully unsaturated ring having 0-1 heteroatoms independently selected from nitrogen wherein said optional substituents on said Ar ring are selected from halogen, —OR°, and —R° wherein R° is selected from hydrogen or C₁₋₄ aliphatic. In another embodiment, said halogen substitutents on said Ar ring are selected from 2-Cl, 3-Cl, 3,4-diCl, 2-F, 3-F, 2-Cl 4-F, or 2,4-diF, and said —OR° substitutents are selected from 2-OH, or 3-OH.

In yet another embodiment, for compounds of formula I or Ia, Z is —C(O)—, V¹, V², and V³ are C(R⁵) wherein R⁵ is hydrogen, R^{x} is hydrogen, R¹ is T-Ar, T is selected from —CH₂—, and Ar is a 9-10 membered optionally substituted, partially unsaturated bicyclic ring having 0-2 heteroatoms independently selected from oxygen.

According to another embodiment, for compounds of formula I or Ia, Z is —C(O)—, V¹, V², and V³ are C(R⁵) wherein R⁵ is hydrogen, R^{x} is hydrogen, R¹ is T-Ar, and T is selected from —CH₂S—.

In another embodiment, for compounds of formula I or Ia, Z is —C(O)—, V¹, V², and V³ are C(R⁵) wherein R⁵is hydrogen, R^{x} is hydrogen, R¹ is T-Ar, T is selected from —CH₂S—, and Ar is a 6 membered optionally substituted, fully unsaturated ring having 0-1 heteroatoms independently selected from nitrogen.

In yet another embodiment, for compounds of formula I or Ia, Z is —C(O)—, V¹, V², and V³ are C(R⁵) wherein R⁵ is hydrogen, R^{x} is hydrogen, R¹ is T-Ar, T is selected from —CH₂S—, and Ar is a 6 membered optionally substituted, fully unsaturated ring having 0-1 heteroatoms independently selected from nitrogen wherein said optional substituents on said Ar ring are selected from halogen, and —R° wherein R° is selected from hydrogen or C₁₋₄ aliphatic. In another embodiment, said halogen substitutents on said Ar ring are selected from 2-Cl, 3-Cl, 4-Cl, 3,4-diCl, 2-F, 3-F, 4-F, 2-Cl-4-F, or 2,4-diF, and said —OR° substitutents are selected from 2-OH, 3-OH, 3-OMe. In another embodiment said Ar substituents include 3-CF₃, 4-CF₃, 3-Me, 4-Me, 2,4-diMe, or 3,4-diMe.

According to another embodiment, for compounds of formula I or Ia, Z is —C(O)—, V¹, V², and V³ are C(R⁵) wherein R⁵ is hydrogen, R^{x} is hydrogen, R¹ is T-Ar, and T is selected from —CH₂N(R)— wherein R is hydrogen.

In another embodiment, for compounds of formula I or Ia, Z is —C(O)—, V¹, V², and V³ are C(R⁵) wherein R⁵ is hydrogen, R^{x} is hydrogen, R¹ is T-Ar, T is selected from —CH₂N(R)—, wherein R is hydrogen and Ar is a 6 membered optionally substituted, fully unsaturated ring having 0-1 heteroatoms independently selected from nitrogen.

In yet another embodiment, for compounds of formula I or Ia, Z is —C(O)—, V¹, V², and V³ are C(R⁵) wherein R⁵ is hydrogen, R^{x} is hydrogen, R¹ is T-Ar, T is selected from —CH₂N(R)—, wherein R is hydrogen and Ar is a 6 membered optionally substituted, fully unsaturated ring having 0-1 heteroatoms independently selected from nitrogen wherein said optional substituents on said Ar ring are selected from halogen, and —R° wherein R° is selected from hydrogen or C₁₋₄ aliphatic. In another embodiment, said halogen substitutents on said Ar ring are selected from 2-Cl, 3-Cl, 4-Cl, 3,4-diCl, 2-F, 3-F, 4-F, 2-Cl-4-F, or 2,4-diF, and said —OR° substitutents are selected from 2-OH, 3-OH, 2-OMe, 3-OMe. In another embodiment said Ar substituents include 3-CF₃, 4-CF₃, 3-Me, 4-Me, 2,4-diMe, or 3,4-diMe.

In another embodiment, for compounds of formula I or Ia, Z is —C(O)—, V¹, V², and V³ are C(R⁵) wherein R⁵ is hydrogen, R^{x} is hydrogen, R¹ is T-Ar, wherein T is as defined in any of the embodiments herein and said optional substituent on said Ar ring is 2-CF₃, 3-CF₃, or 4-CF₃.

According to another embodiment, the present invention relates to a compound of formula Ib: 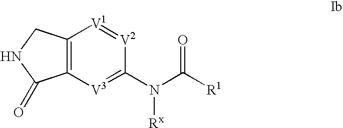
or a pharmaceutically acceptable salt thereof, wherein V¹, V², V³, and R¹ are as defined above for compounds of formula I.

One aspect of the present invention relates to a compound of formula Ib wherein R^{x} is hydrogen.

Another aspect of the present invention relates to a compound of formula Ib wherein R^{x} is T-Ar.

Yet another aspect of the present invention relates to a compound of formula Ib wherein R^{x} and R¹ are taken together to form an optionally substituted 5-7 membered saturated or partially unsaturated ring having 1-4 heteroatoms independently selected from nitrogen, oxygen, or sulfur.

According to one embodiment, the ring formed by the R^{x} and R¹ groups of formula Ib is a 5-6 membered saturated ring having 1-2 heteroatoms independently selected from nitrogen, oxygen, or sulfur.

According to another embodiment, the present invention relates to a compound of formula Ib wherein V¹ is N, V² is C(R⁵), and V³ is C(R⁵).

Other compounds of formula Ib include those wherein V¹ is C(R⁵), V² is C(R⁵), and V³ is C(R⁵) or N.

Another embodiment of the present invention relates to a compound of formula Ib wherein V¹ is C(R⁵), V² is C(R⁵) or N, and V³ is N.

Another embodiment of the present invention relates to a compound of formula Ib wherein V¹ is CH, V² is CH, and V³ is CH.

According to another embodiment, the present invention relates to a compound of formula Ib wherein V¹ is CH, and V² and V³ are each N.

When R¹ of formula Ib is T-Ar, such Ar groups include an optionally substituted ring selected from a 5-6 membered saturated, partially unsaturated, or fully unsaturated monocyclic ring having 0-4 heteroatoms independently selected from nitrogen, oxygen, or sulfur, or a 9-10 membered saturated, partially unsaturated, or fully unsaturated bicyclic ring having 0-4 heteroatoms independently selected from nitrogen, oxygen, or sulfur. Examples of such monocyclic rings include phenyl, pyridyl, pyrimidinyl, pyridonyl, furanyl, tetrazolyl, thienyl, cyclopentyl, cyclohexyl, and cycloheptyl. Examples of such bicyclic rings include benzo[1,3]dioxolyl, indan-1-onyl, naphthyl, benzothiophenyl, 2,3-dihydro-1H-isoindolyl, indanyl, benzofuranyl, and indolyl.

When present, substituents on the Ar ring of R¹ include R°, halogen, oxo, OR°, phenyl, optionally substituted dialkylamino, haloalkyl, C(O)R°, NHC(O)R, or SR°. Examples of such substituents include chloro, bromo, fluoro, OH, OMe, NHC(O)CH₃, OEt, C(O)phenyl, Ophenyl, N(CH₂CH₂Cl)₂, N(Me)₂, CF₃, and SCF₃. Other examples of Ar groups of formula Ib also include those shown in Table 1 below.

When the R¹ group of formula Ib is T-C(R)(T-Ar)R², R² groups include R′, Q-OR³, Q-N(R⁴)₂, Ar¹, N(R)C(O)Q-N(R⁴)₂, and N(R)Q-N(R⁴)₂. Examples of such R² groups include CH₂OH, OH, NH₂, CH₂NH₂, CH₂NHMe, CH₂N(Me)₂, CH₂CH₂NH₂, CH₂CH₂NHMe, CH₂CH₂N(Me)₂, CH₂C(Me)₂NH₂, CH₂C(Me)₂CHMe, NHCO₂t-butyl, phenyl, cyclopentyl, methyl, ethyl, isopropyl, cyclopropyl, NH(CH₂)₃NH₂, NH(CH₂)₂NH₂, NH(CH₂)₂NHEt, NHCH₂pyridyl, NHSO₂phenyl, NHC(O)CH₂C(O)Ot-butyl, NHC(O)CH₂NH₃, and NHCH₂-imidazol-4-yl.

According to another embodiment, the R² group of formula Ib is selected from OH, NH₂, CH₂NH₂, CH₂NHMe, CH₂N(Me)₂, CH₂CH₂NH₂, CH₂CH₂NHMe, CH₂CH₂N(Me)₂, CH₂C(Me)₂NH₂, CH₂C(Me)₂CHMe, NHCO₂t-butyl, phenyl, NH(CH₂)₃NH₂, NH(CH₂)₂NH₂, NH(CH₂)₂NHEt, NHCH₂pyridyl, NHSO₂phenyl, NHC(O)CH₂C(O)Ot-butyl, NHC(O)CH₂NH₃, and NHCH₂-imidazol-4-yl.

According to another embodiment, the R² group of formula Ib is selected from CH₂CH₂NH₂.

When present, rings formed by the R and R² moieties of the T-C(R)(T-Ar)R² group of R¹ are selected from a 5-6 membered saturated ring having 0-2 heteroatoms independently selected from nitrogen, oxygen, or sulfur. Examples of such rings formed by R and R² include piperidinyl, pyrrolidinyl, piperazinyl, morpholinyl, and thiomorpholinyl.

When the R¹ group of formula Ib is T-C(R)(T-Ar)R², Ar groups of the T-C(R)(T-Ar)R² moiety are selected from an optionally substituted 5-6 membered saturated, partially unsaturated, or fully unsaturated monocyclic ring having 0-4 heteroatoms independently selected from nitrogen, oxygen, or sulfur, or an optionally substituted 9-10 membered saturated, partially unsaturated, or fully unsaturated bicyclic ring having 0-4 heteroatoms independently selected from nitrogen, oxygen, or sulfur. Examples of such monocyclic rings include phenyl, pyridyl, furanyl, pyridone, and thienyl. Examples of such bicyclic rings include benzo[1,3]dioxolyl, naphthyl, indanyl, and indolyl. When present, substituents on the Ar ring of the T-C(R)(T-Ar)R² group of R¹ include R°, halogen, OR°, phenyl, N(R°)₂, NHC(O)R°, or SR°. Examples of such groups include fluoro, chloro, bromo, CF₃, OH, OMe, OPh, OCH₂Ph, SMe, NH₂, NHC(O)Me, methyl, ethyl, isopropyl, isobutyl, and cyclopropyl.

R⁵ groups of formula Ib, when present, are selected from halogen, R, and Ar¹. Other R⁵ groups of formula Ib, when present, are selected from halogen, optionally substituted C₁₋₄ aliphatic, or an optionally substituted 5-6 membered saturated, partially unsaturated, or fully unsaturated monocyclic ring having 0-4 heteroatoms independently selected from nitrogen, oxygen, or sulfur. Examples of such groups include chloro, bromo, methyl, ethyl, t-butyl, cyclopropyl, isopropyl, phenyl, and pyridyl.

Representative compounds of formula I are set forth in Table 1 below. In certain embodiments, the variables are defined as depicted in the Table 1 compounds.

TABLE 1

| Cmpd No. | Structure |
|---|---|
| I-1 | 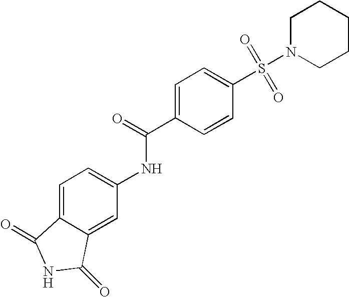 |
| I-2 | 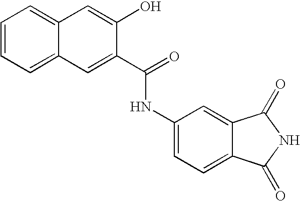 |
| I-3 | 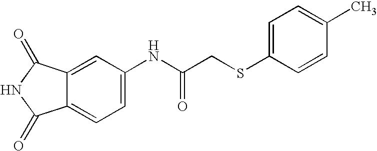 |
| I-4 | 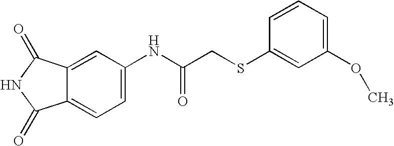 |
| I-5 | 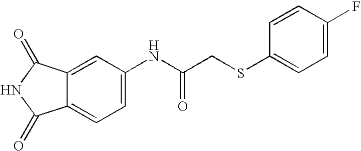 |
| I-6 | 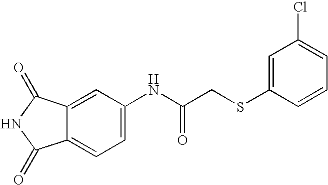 |
| I-7 | 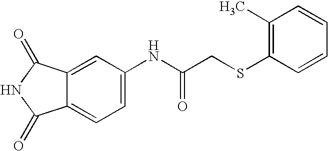 |
| I-8 | 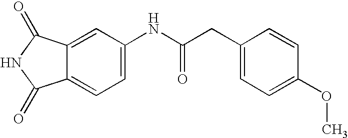 |
| I-9 | 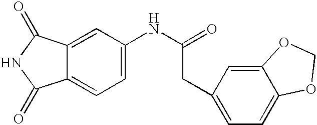 |
| I-10 | 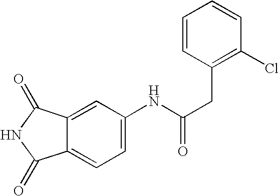 |
| I-11 | 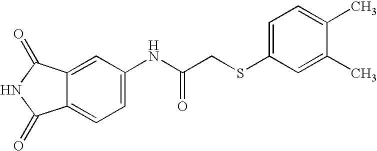 |
| I-12 | 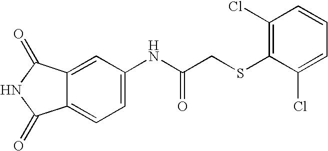 |
| I-13 | 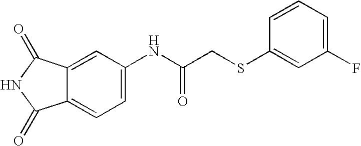 |
| I-14 | 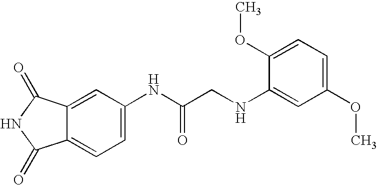 |
| I-15 | 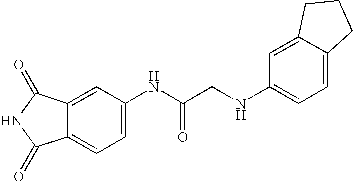 |
| I-16 | 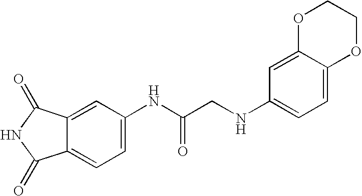 |
| I-17 | 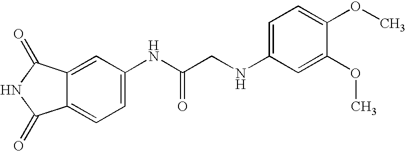 |
| I-18 | 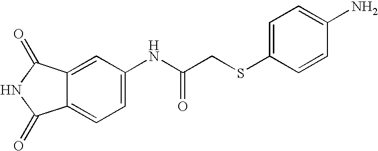 |
| I-19 | 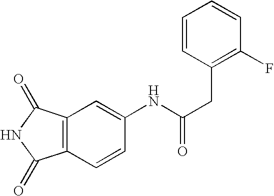 |
| I-20 | 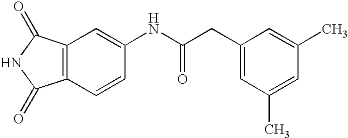 |
| I-21 | 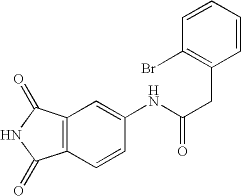 |
| I-22 | 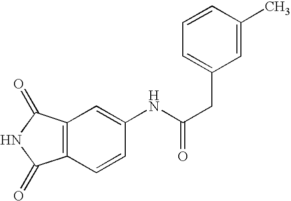 |
| I-23 | 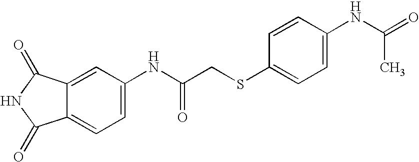 |
| I-24 | 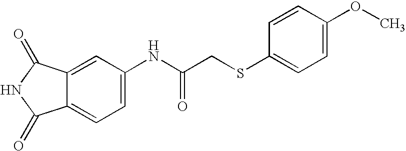 |
| I-25 | 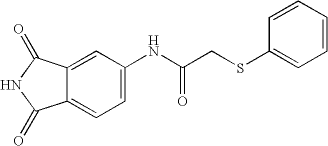 |
| I-26 | 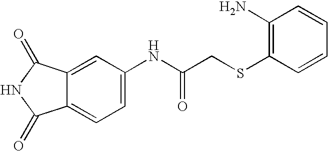 |
| I-27 | 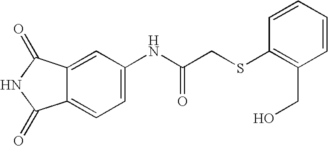 |
| I-28 | 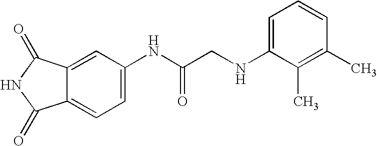 |
| I-29 | 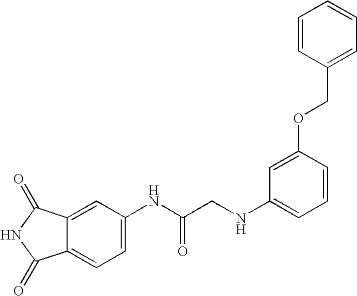 |
| I-30 | 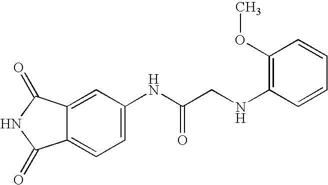 |
| I-31 | 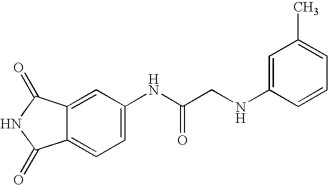 |
| I-32 | 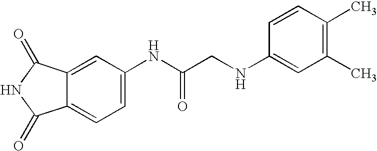 |
| I-33 | 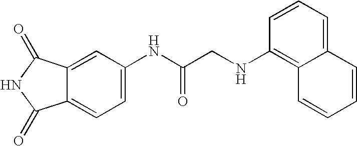 |
| I-34 | 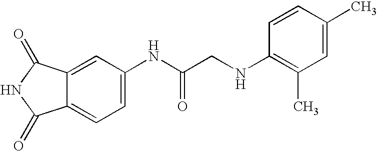 |
| I-35 | 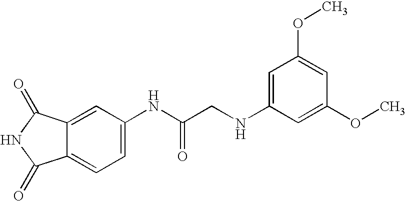 |
| I-36 | 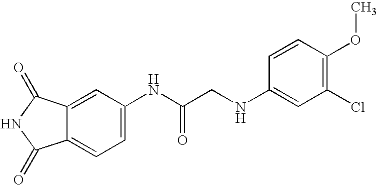 |
| I-37 | 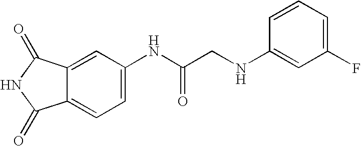 |
| I-38 | 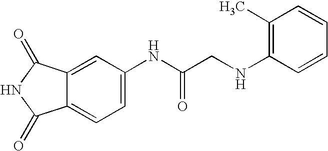 |
| I-39 | 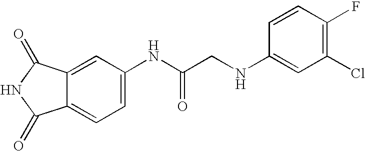 |
| I-40 | 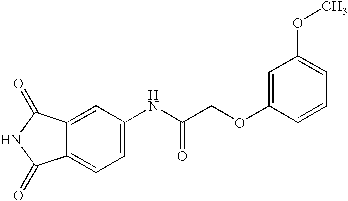 |
| I-45 | 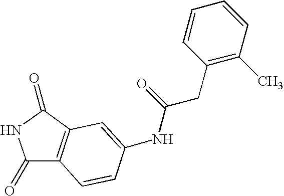 |
| I-46 | 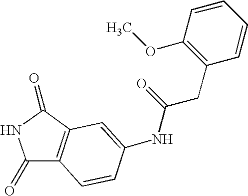 |
| I-47 | 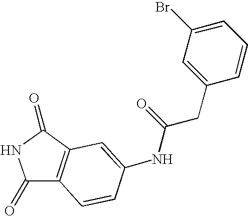 |
| I-48 | 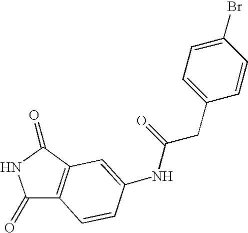 |
| I-49 | 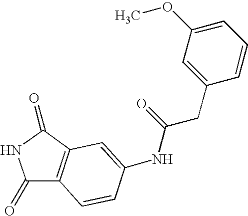 |
| I-50 | 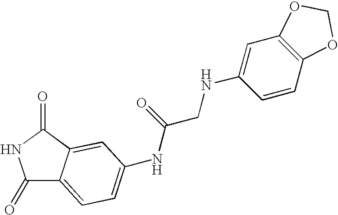 |
| I-51 | 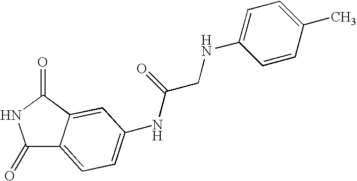 |
| I-52 | 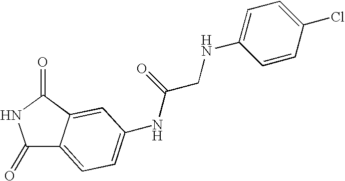 |
| I-53 | 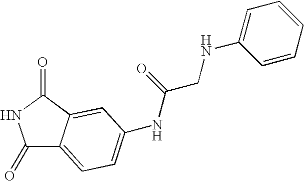 |
| I-54 | 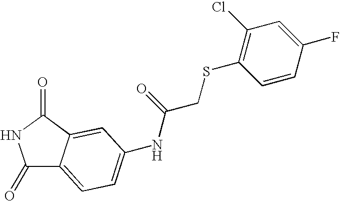 |
| I-55 | 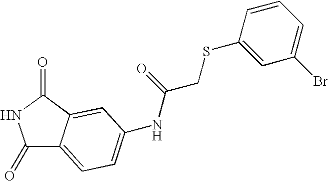 |
| I-56 | 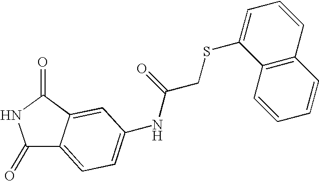 |
| I-57 | 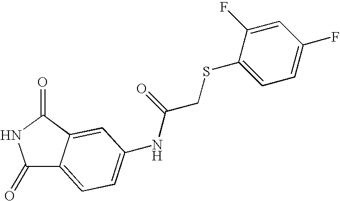 |
| I-58 | 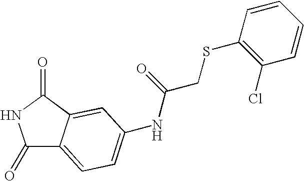 |
| I-59 | 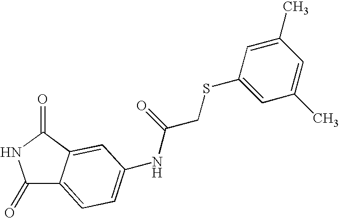 |
| I-60 | 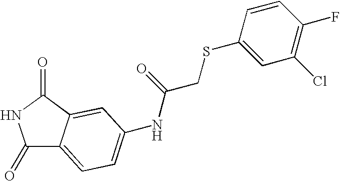 |
| I-61 | 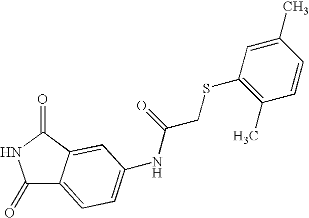 |
| I-62 | 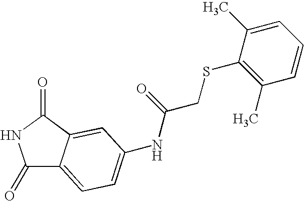 |
| I-63 | 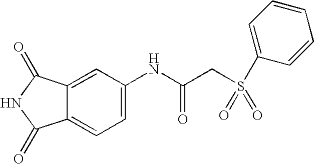 |
| I-64 | 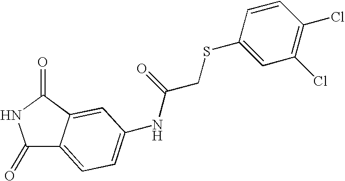 |
| I-65 | 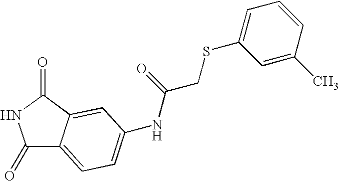 |
| I-66 | 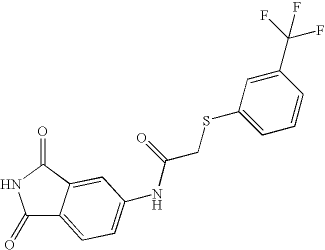 |
| I-67 | 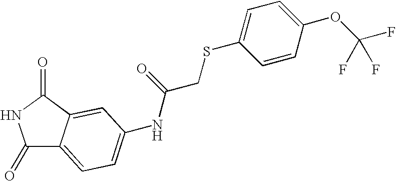 |
| I-68 | 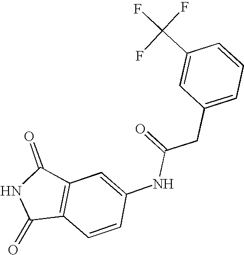 |
| I-69 | 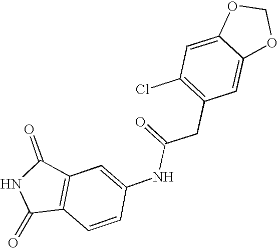 |
| I-70 | 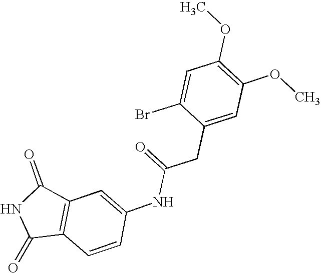 |
| I-71 | 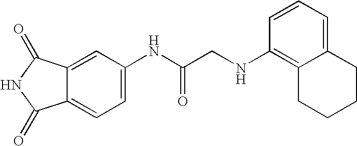 |
| I-72 | 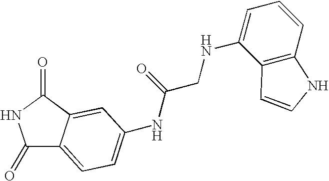 |
| I-73 | 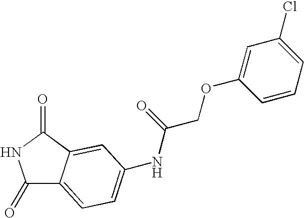 |
| I-74 | 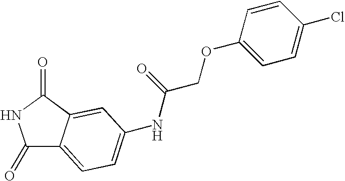 |
| I-75 | 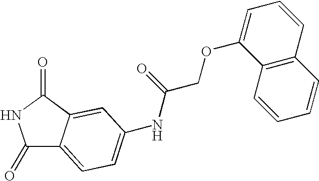 |
| I-76 | 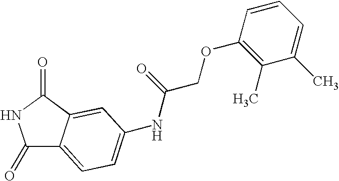 |
| I-77 | 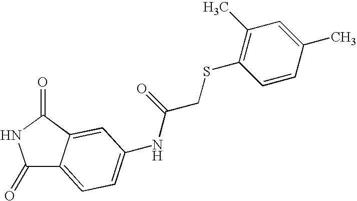 |
| I-78 | 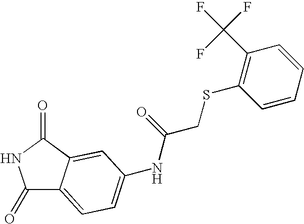 |
| I-79 | 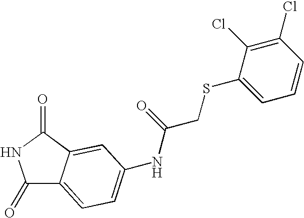 |
| I-80 | 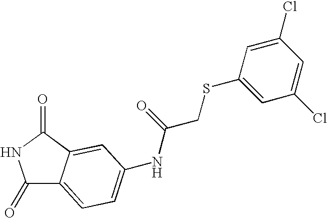 |
| I-81 | 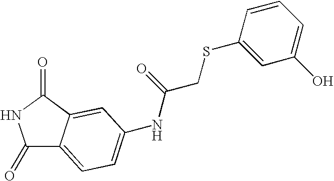 |
| I-82 | 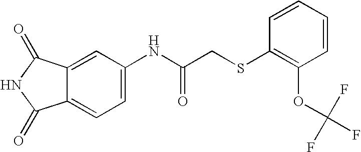 |
| I-83 | 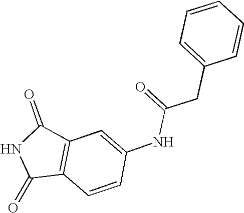 |
| I-84 | 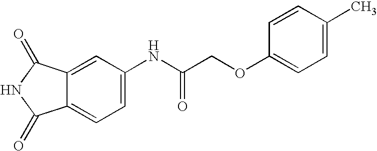 |
| I-85 | 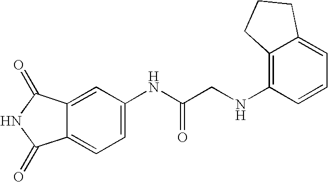 |
| I-86 | 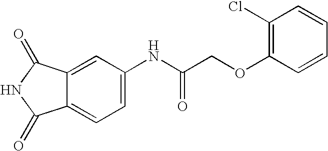 |
| I-87 | 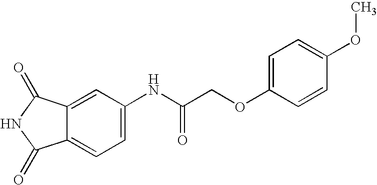 |
| I-88 | 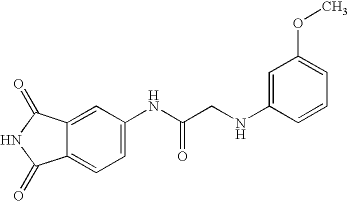 |
| I-89 | 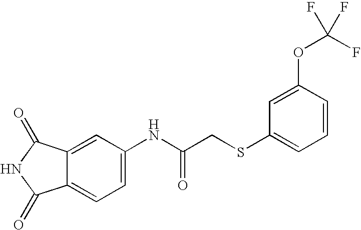 |
| I-90 | 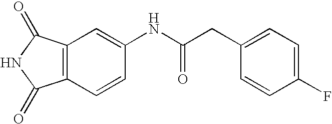 |
| I-91 | 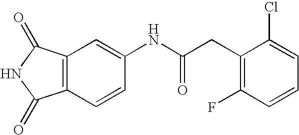 |
| I-92 | 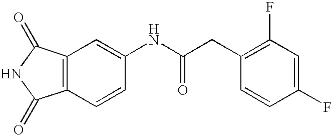 |
| I-93 | 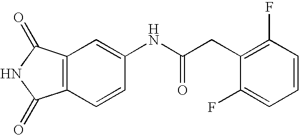 |
| I-94 | 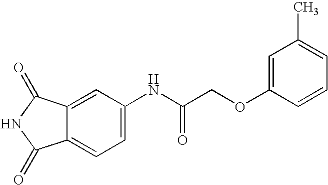 |
| I-95 | 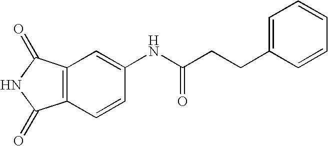 |
| I-96 | 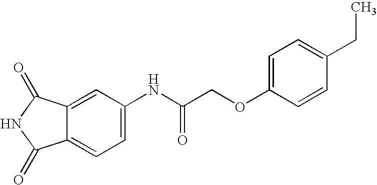 |
| I-97 | 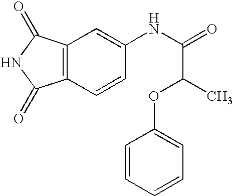 |
| I-98 | 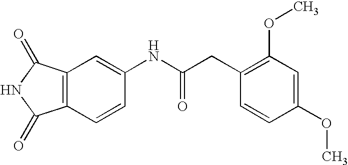 |
| I-99 | 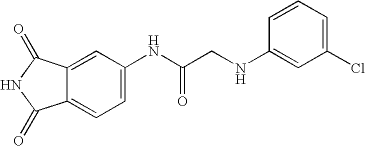 |
| I-100 | 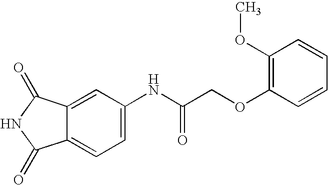 |
| I-101 | 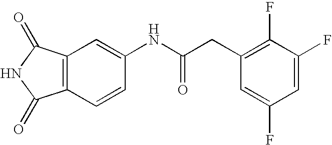 |
| I-102 | 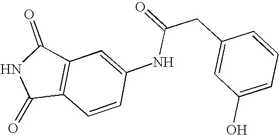 |
| I-103 | 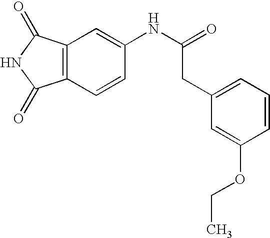 |
| I-104 | 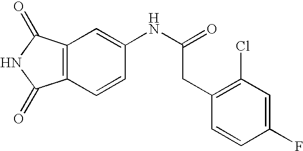 |
| I-105 | 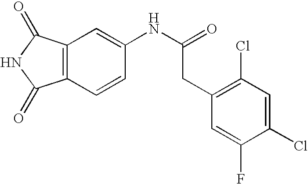 |
| I-106 | 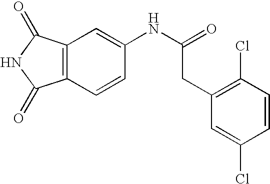 |
| I-107 | 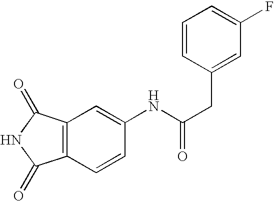 |
| I-108 | 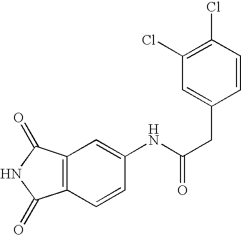 |
| I-109 | 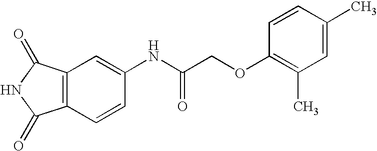 |
| I-110 | 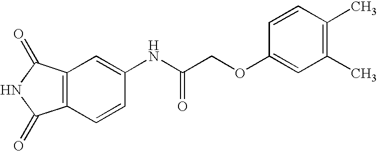 |
| I-111 | 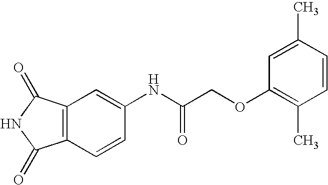 |
| I-112 | 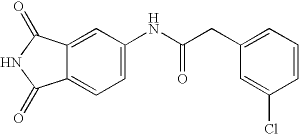 |
| I-113 | 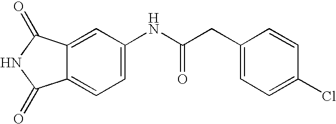 |
| I-114 | 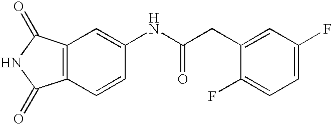 |
| I-115 | 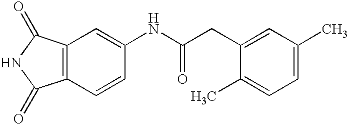 |
| I-116 | 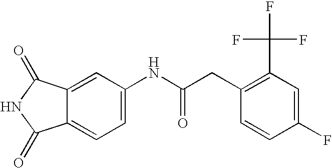 |
| I-117 | 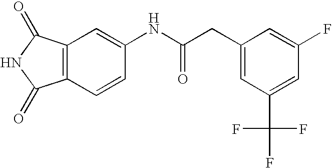 |
| I-118 | 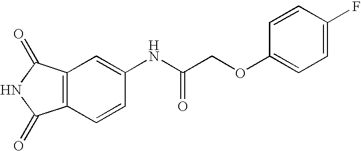 |
| I-119 | 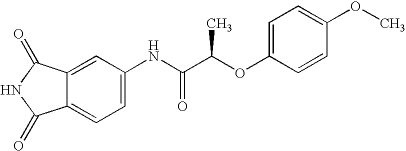 |
| I-120 | 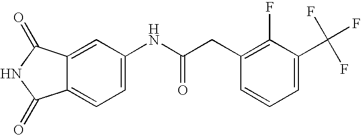 |
| I-121 | 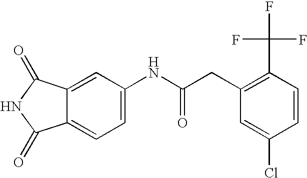 |
| I-122 | 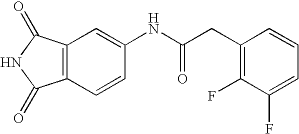 |
| I-123 | 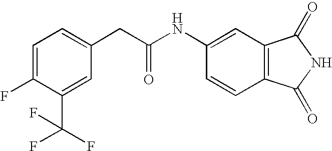 |
| I-124 | 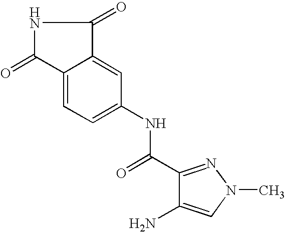 |
| I-125 | 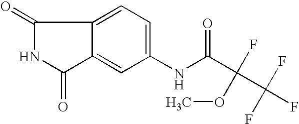 |
| I-126 | 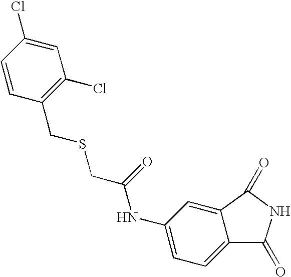 |
| I-127 | 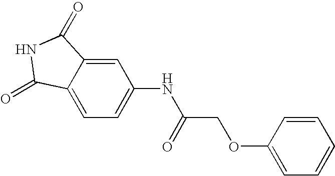 |
| I-128 | 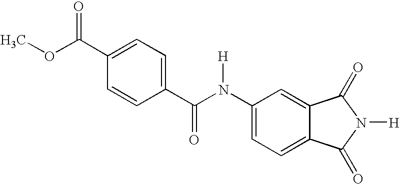 |
| I-129 | 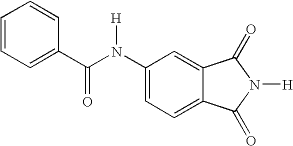 |
| I-130 | 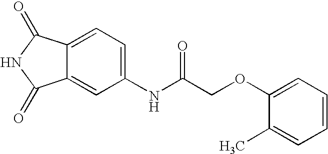 |
| I-131 | 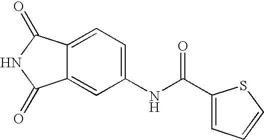 |
| I-132 | 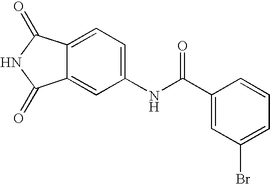 |
| I-133 | 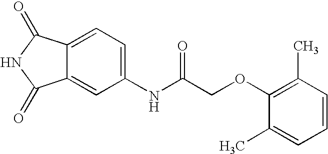 |
| I-134 | 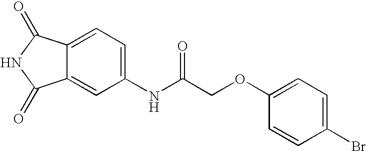 |
| I-135 | 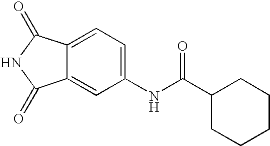 |
| I-136 | 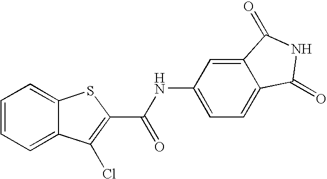 |
| I-137 | 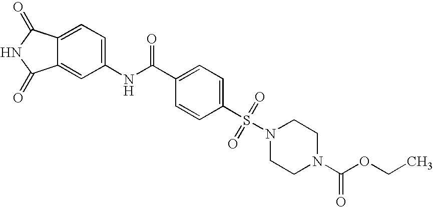 |
| I-138 | 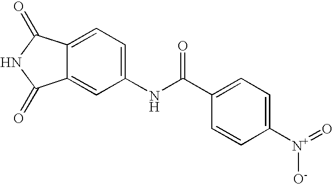 |
| I-139 | 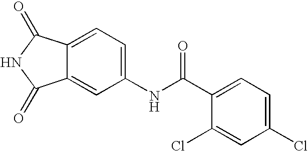 |
| I-140 | 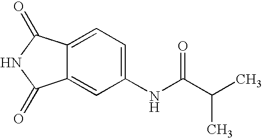 |

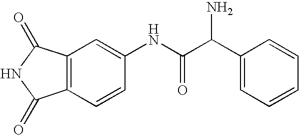

I-141

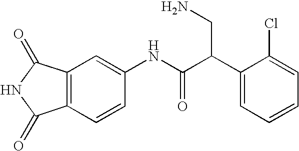

I-142

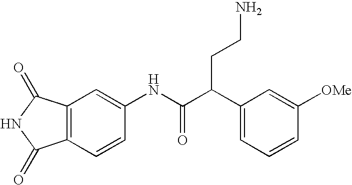

I-143

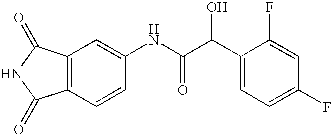

I-144

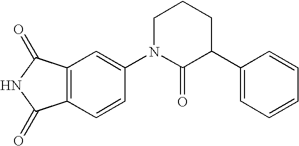

I-145

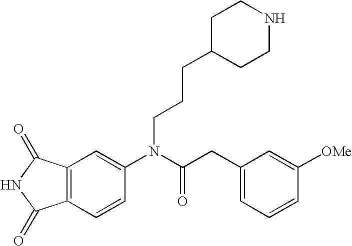

I-146

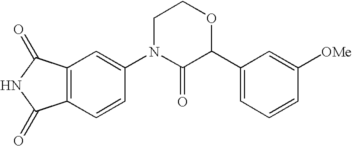

I-147

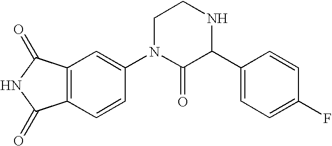

I-148

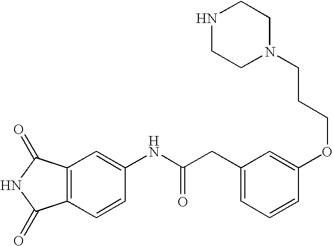

I-149

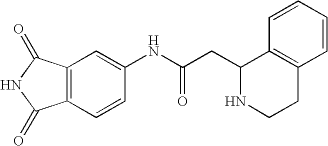

I-150

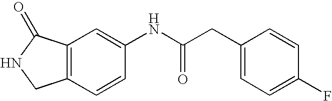

I-151

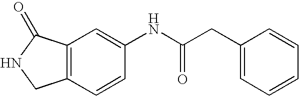

I-152

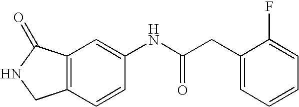

I-153

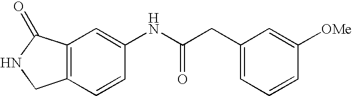

I-154

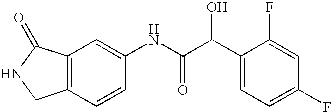

I-155

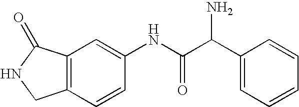

I-156

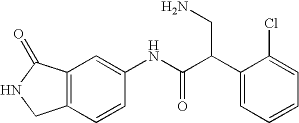

I-157

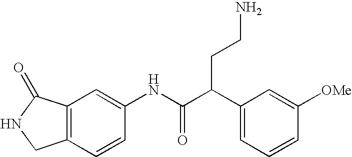

I-158

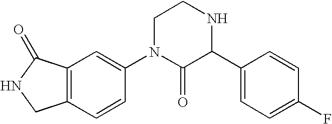

I-159

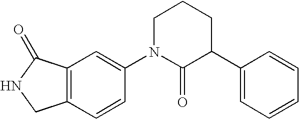

I-160

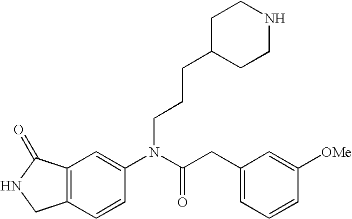

I-161

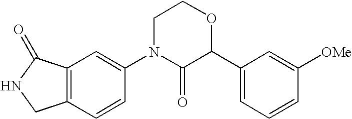

I-162

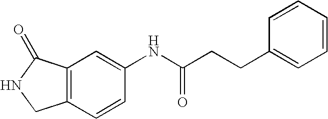

I-163

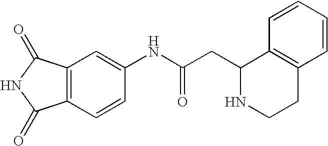

I-164

The compounds of the present invention may be prepared as illustrated by the Schemes I-V and by general methods known to those of ordinary skill in the art.

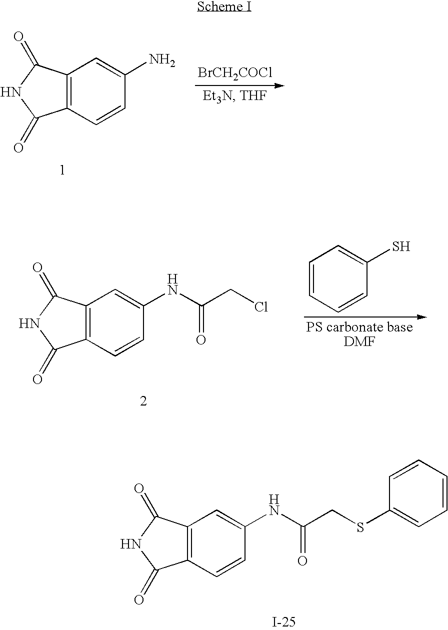

Using compound I-25 to illustrate, Scheme I above shows a general method for preparing the phthalimide compounds of the present invention where T is a C₁₋₆ alkylidene chain wherein one methylene unit of T is repaced by —S—. The amino-phthalimide compound 1 is treated with bromoacetyl chloride to form the chloro intermediate (2). Intermediate 2 is then treated with thiophenol to form compound 1-25. One of ordinary skill in the art would recognize that intermediate 2 can be treated with a variety of reagents to form compounds of the present invention.

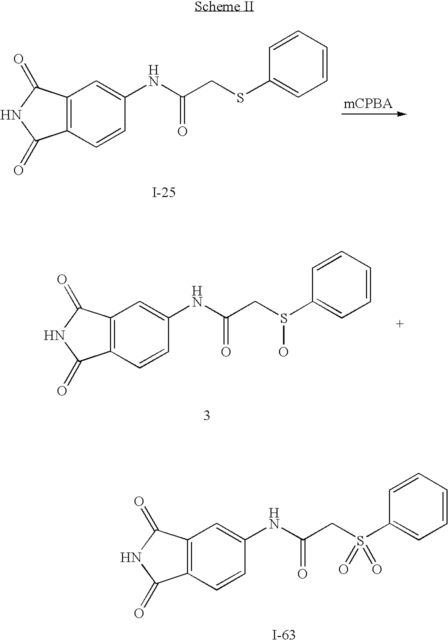

Scheme II above shows a general method for preparing certain compounds, such as I-63, of the present invention. The sulfur moiety of compound I-25 is oxidized by treatment with mCPBA to form the sulphonyl compound I-63.

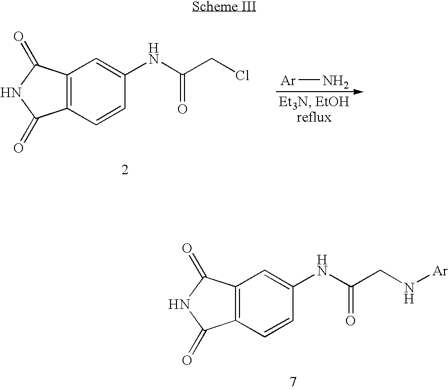

Scheme III above shows a method for preparing compounds of formula I where T is an alkylidene chain wherein one methylene unit of T is replaced by —NH—. The chloro intermediate 2 is treated with Ar—NH₂ to form compound 7.

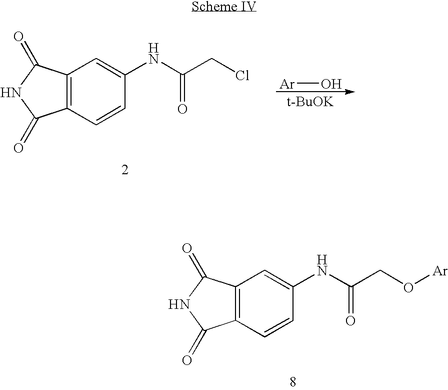

Scheme IV above shows a method for preparing compounds of formula I where T is an alkylidene chain wherein one methylene unit of T is replaced by —O—. The chloro intermediate 2 is treated with Ar—OH in the presence of base to form compound 8.

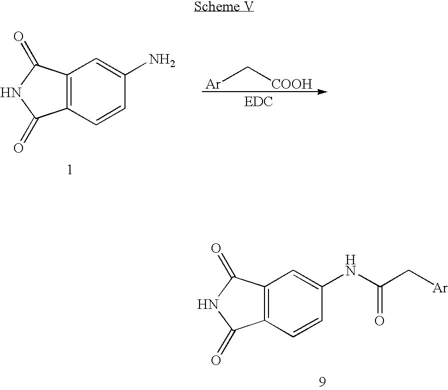

Scheme V above shows a general method for preparing the amide compounds of the present invention. Starting amine (1) is treated with a carboxylic acid to form the amide (9). One of ordinary skill in the art would recognize that a wide variety of carboxylic acids are amenable to this reaction under a wide variety of coupling conditions including, but not limited to, the EDC reagent depicted.

The general synthetic methods described above in Schemes I-V may also be used to prepare compounds of formula I wherein Z is —CH₂—.

Accordingly, another embodiment of this invention provides processes for preparing the compounds of this invention according to the methods of Schemes I-V. It should be recognized that other general methods may be employed consistent with the above schemes.

Analytical data for certain compounds of the present invention was collected and recorded as follows. The LC/MS method used a Hypersil BDS C18 5 micron 2.1×50 mm column with a flow rate of 1.0 ml/min using a 0-95% MeCN (0.1% TFA) in H₂O (0.1% TFA) gradient over a run time of 2.39 minutes. Proton NMR was collected at 400 MHz using deuterated DMSO as solvent. Table 2 below depicts exemplary LC (liquid chromatography) mass spectral data (LC/MS) and ¹H-NMR data for certain compounds of the present invention:

| | LC/MS | | |
|---|---|---|---|
| Cmpd. | Ret. Time | LC/MS | ¹H-NMR (ppm) |
| No. | (min) | (M + H) | [400 MHz in DMSO-d₆] |
| I-3 | 1.29 | 327.00 | 11.21(1H, s), 8.11(1H, d, 1.3Hz), 7.82(1H, |
| | | | dd, 8.2Hz, 1.7Hz), 7.77(1H, d, 8.3Hz), 7.32(2H, |
| | | | d, 8.1Hz), 7.15(2H, d, 8.1Hz), 3.85(2H, |
| | | | s), 2.27(3H, s) |
| I-4 | 1.22 | 343.00 | 8.25(1H, d, 1.5Hz), 8.01(1H, dd, 8.1Hz, |
| | | | 1.8Hz), 7.95,(1H, d, 8.1Hz), 7.42(1H, t, |
| | | | 8.1Hz), 7.17-7.12(2H, m), 6.96(1H, dd, |
| | | | 8.1Hz, 2.5Hz), 4.12(2H, s), 3.91(3H, s) |
| I-5 | 1.23 | 331.00 | 10.72(1H, s), 8.07(1H, d, 1.52Hz), 7.78(1H, |
| | | | dd, 8.1Hz, 1.8Hz), 7.74(1H, d, |
| | | | 8.1Hz), 7.49-7.43(2H, m), 7.20-7.14(2H, |
| | | | m), 3.84(2H, s) |
| I-6 | 1.33 | 347.00 | 11.00(1H, s), 10.59(1H, s), 7.87(1H, d, |
| | | | 1.3Hz), 7.60(1H, dd, 8.1Hz, 1.8Hz), 7.55(1H, |
| | | | d, 8.1Hz), 7.27(1H, br s), 7.15-7.02(3H, |
| | | | m), 3.77(2H, s) |
| I-7 | 1.29 | 327.00 | 11.24(1H, s), 10.81(1H, s), 8.12(1H, d, |
| | | | 1.5Hz), 7.85(1H, dd, 8.1Hz, 1.8Hz), 7.80(1H, |
| | | | d, 8.1Hz), 7.41(1H, d, 7.0Hz), 7.27-7.13(3H, |
| | | | m), 3.92(2H, s), 2.35(3H, s) |
| I-8 | 1.13 | 311.00 | 11.26(1H, s), 10.77(1H, s), 8.21(1H, dd, |
| | | | 8.3Hz, 1.7Hz), 7.93(1H, d, 8.3Hz), 7.32(2H, |
| | | | d, 8.8Hz), 6.96(2H, d, 8.6Hz), 3.79(3H, |
| | | | s), 3.70(2H, s) |
| I-9 | 1.12 | 325.00 | 11.30(1H, br s), 10.76(1H, s), 8.23(1H, d, |
| | | | 1.5Hz), 7.95(1H, dd, 8.3Hz, 1.8Hz), 7.85(1H, |
| | | | d, 8.3Hz), 7.00(1H, d, 1.5Hz), 6.95(1H, |
| | | | d, 7.8Hz), 6.88(1H, dd, 7.8Hz, |
| | | | 1.7Hz), 6.07(2H, s), 3.71(2H, s) |
| I-10 | 1.21 | 315.00 | 11.27(1H, br s), 10.89(1H, s), 8.21(1H, d, |
| | | | 1.52Hz), 7.93(1H, dd, 8.1Hz, 1.77Hz), 7.84(1H, |
| | | | d, 8.1Hz), 7.54-7.48(2H, m), 7.42-7.37(2H, |
| | | | m), 3.98(2H, s) |
| I-11 | 1.34 | 341.00 | 11.23(1H, br s), 10.73(1H, s), 8.10(1H, d, |
| | | | 1.3Hz), 7.82(1H, dd, 8.1Hz, 1.8Hz), 7.77(1H, |
| | | | d, 8.1Hz), 7.22(1H, s), 7.15(1H, dd, |
| | | | 7.8Hz, 1.8Hz), 7.09(1H, d, 7.8Hz), 3.84(2H, |
| | | | s), 2.18(3H, s), 2.17(3H, s) |
| I-12 | 1.32 | 381.00 | 11.21(1H, s), 10.71(1H, s), 8.03(1H, br s), |
| | | | 7.78-7.72(2H, m), 7.58(1H, d, 8.1Hz), |
| | | | 7.43(2H, t, 7.6Hz), 3.71(2H, s) |
| I-13 | 1.24 | 331.00 | 11.03(1H, s), 10.62(1H, s), 7.89(1H, d, |
| | | | 1.5Hz), 7.62(1H, dd, 8.3Hz, 1.8Hz), 7.57(1H, |
| | | | d, 8.3Hz), 7.19-7.13(1H, m), 7.09(1H, |
| | | | dt, 10.0Hz, 2.2Hz), 7.03-6.91(1H, m), |
| | | | 6.83(1H, td, 8.1Hz, 2.5Hz), 3.79(2H, s) |
| I-14 | 1.16 | 356.00 | 11.21(1H, s), 10.64(1H, s), 8.15(1H, d, |
| | | | 1.8Hz), 7.88(1H, dd, 8.1Hz, 1.8Hz), 7.77(1H, |
| | | | d, 8.1Hz), 6.73(1H, d, 8.7Hz), 6.13(1H, |
| | | | dd, 8.7, 2.8Hz), 6.03(1H, d, 2.8Hz), |
| | | | 5.41(1H, t, 5.8Hz), 3.96(2H, d, 5.8Hz), |
| | | | 3.76(3H, s), 3.62(3H, s) |
| I-15 | 1.18 | 336.00 | 11.26(1H, s), 10.58(1H, s), 8.24(1H, d, |
| | | | 1.8Hz), 7.97(1H, dd, 8.1Hz, 1.8Hz), 7.83(1H, |
| | | | d, 8.1Hz), 7.00(1H, d, 8.1Hz), 6.55(1H, |
| | | | d, 2.0Hz), 6.46(1H, dd, 8.1Hz, |
| | | | 2.0Hz), 5.87(1H, t, 6.3Hz), 3.95(2H, d, 6.3Hz), |
| | | | 2.81-2.73(4H, m), 2.03-1.96(2H, m) |
| I-16 | 0.97 | 354.00 | 11.22(1H, s), 10.54(1H, s), 8.18(1H, d, |
| | | | 1.6Hz), 7.91(1H, dd, 8.1Hz, 1.6Hz), 7.77(1H, |
| | | | d, 8.1Hz), 6.62(1H, d, 8.6Hz), 6.13(1H, |
| | | | dd, 8.6Hz, 2.6Hz), 6.10(1H, d, |
| | | | 2.6Hz), 5.71(1H, t, 6.2Hz), 4.16-4.08(4H, |
| | | | m), 3.84(2H, d, 6.2Hz) |
| I-17 | 0.90 | 356.00 | 11.01(1H, s), 10.44(1H, s), 8.06(1H, d, |
| | | | 1.8Hz), 7.80(1H, dd, 8.1Hz, 1.8Hz), 7.66(1H, |
| | | | d, 8.1Hz), 6.62(1H, d, 8.7Hz), 6.27(1H, |
| | | | d, 2.5Hz), 5.93(1H, dd, 8.7Hz, 2.5Hz), |
| | | | 5.58(1H, t, 6.0Hz), 3.78(2H, d, 6.0Hz), |
| | | | 3.58(3H, s), 3.50(3H, s) |
| I-18 | 0.81 | 328.00 | 11.20(1H, br s), 10.56(1H, s), 8.10(1H, d, |
| | | | 1.52Hz), 7.81(1H, dd, 8.3Hz, 1.8Hz), 7.76(1H, |
| | | | d, 8.1Hz), 7.15(2H, d, 8.6Hz), 6.50(2H, |
| | | | d, 8.6Hz), 5.30(2H, s), 3.58(2H, s) |
| I-19 | 1.23 | 299.00 | 10.67(1H, s), 7.99(1H, d, 1.5Hz), 7.72(1H, |
| | | | dd, 8.3Hz, 1.8Hz), 7.63(1H, d, |
| | | | 8.3Hz), 7.26(1H, dt, 7.3Hz, 1.8Hz), 7.22-7.15(2H, |
| | | | m), 7.07-7.00(2H, m), 3.67(2H, |
| | | | s) |
| I-20 | 1.39 | 309.00 | 10.80(1H, s), 8.24(1H, d, 1.5Hz), 7.95(1H, |
| | | | dd, 8.3Hz, 1.8Hz), 7.85(1H, d, |
| | | | 8.1Hz), 7.03(2H, s), 6.98(1H, s), 3.71(2H, |
| | | | s), 2.34(6H, s) |
| I-21 | 1.30 | 359.00 | 10.62(1H, s), 7.93(1H, d, 1.5Hz), 7.65(1H, |
| | | | dd, 8.3Hz, 1.5Hz), 7.56(1H, d, |
| | | | 8.3Hz), 7.41(1H, d, 8.1Hz), 7.22(1H, dd, |
| | | | 7.6Hz, 1.3Hz), 7.16(1H, dt, 7.3Hz, 1.0Hz), |
| | | | 7.02(1H, dt, 7.8Hz, 1.3Hz), 3.71(2H, s) |
| I-22 | 1.31 | 294.00 | 10.50(1H, s), 7.91(1H, d, 1.7Hz), 7.63(1H, |
| | | | dd, 8.1Hz, 1.8Hz), 7.52(1H, d, |
| | | | 8.1Hz), 6.98(1H, t, 7.6Hz), 6.92-6.86(2H, |
| | | | m), 6.83(1H, d, 7.3Hz), 3.43(2H, s), 2.05(3H, |
| | | | s) |
| I-23 | 1.07 | 370.00 | 11.23(1H, s), 10.72(1H, s), 9.99(1H, s), |
| | | | 8.09(1H, d, 1.3Hz), 7.83-7.76(2H, m), |
| | | | 7.54(2H, d, 9.0Hz), 7.37(2H, d, 9.0Hz), |
| | | | 3.82(2H, s), 2.03(3H, s) |
| I-24 | 1.28 | 343.00 | 11.17(1H, s), 10.62(1H, s), 8.02(1H, d, |
| | | | 1.3Hz), 7.74-7.69(2H, m), 7.33(2H, d, |
| | | | 9.0Hz), 6.84(2H, d, 9.0Hz), 3.68(2H, s), |
| | | | 3.66(3H, s) |
| I-25 | 1.28 | 313.00 | 10.73(1H, s), 8.04(1H, d, 1.5Hz), 7.76(1H, |
| | | | dd, 1.8Hz, 8.5Hz), 7.69(1H, d, |
| | | | 8.0Hz), 7.34(2H, dd, 8.0Hz, 1.3Hz), 7.26(2H, |
| | | | t, 8.0Hz), 7.15(1H, t, 7.5Hz), 3.86(2H, |
| | | | s) |
| I-26 | 1.03 | 328.00 | 10.64(1H, s), 8.10(1H, d, 1.3Hz), 7.81-7.76(2H, |
| | | | m), 7.27(1H, dd, 8.0Hz, 1.5Hz), |
| | | | 7.04(1H, dt, 1.5Hz, 7.0Hz), 6.71(1H, dd, |
| | | | 8.0Hz, 1.3Hz), 6.48(1H, ddd, 1.3Hz, |
| | | | 7.3Hz, 7.3Hz), 5.46(2H, s), 3.57(2H, s) |
| I-27 | 1.10 | 343.00 | 11.29(1H, s), 10.85(1H, s), 8.16(1H, d, |
| | | | 1.5Hz), 7.89-7.82(2H, m), 7.53-7.49(2H, |
| | | | m), 7.32-7.30(2H, m), 5.33(1H, t, 5.5Hz), |
| | | | 4.65(2H, d, 5.5Hz), 3.95(2H, s). |
| I-28 | 1.77 | 324.00 | 11.22(1H, s), 10.55(1H, s), 8.17(1H, d, |
| | | | 1.5Hz), 7.91(1H, dd, 8.0Hz, 1.5Hz), 7.77(1H, |
| | | | d, 8.0Hz), 6.88(1H, t, 7.5Hz), 6.48(1H, |
| | | | d, 7.5Hz), 6.03(1H, d, 7.5Hz), 5.35(1H, |
| | | | t, 6.0Hz), 3.97(2H, d, 6.0Hz), 2.21(3H, |
| | | | s), 2.08(3H, s). |
| I-29 | 1.89 | 402.00 | 11.23(1H, s), 10.59(1H, s), 8.17(1H, d, |
| | | | 1.5Hz), 7.91(1H, dd, 1.8Hz, 8.0Hz), 7.77(1H, |
| | | | d, 8.0Hz), 7.42-7.28(6H, m), 6.99(1H, |
| | | | t, 8.0Hz), 6.28-6.21(3H, m), 5.01(2H, |
| | | | s), 3.93(2H, s). |
| I-30 | 1.69 | 326.00 | 10.70(1H, s), 8.22(1H, d, 1.5Hz), 7.95(1H, |
| | | | dd, 1.8Hz, 8.0Hz), 7.83(1H, d, |
| | | | 8.0Hz), 6.91(1H, dd, 1.3Hz, 8.0Hz), 6.83(1H, |
| | | | ddd, 1.3Hz, 7.5Hz, 7.5Hz), 6.66(1H, |
| | | | ddd, 1.5Hz, 7.5Hz, 7.8Hz), 6.50(1H, dd, |
| | | | 1.5Hz, 7.8Hz), 5.43(1H, t, 6.0Hz), 4.04(2H, |
| | | | d, 6. |
| I-31 | 1.68 | 310.00 | 11.23(1H, s), 10.58(1H, s), 8.17(1H, d, |
| | | | 1.5Hz), 7.91(1H, dd, 1.8Hz, 8.0Hz), 7.77(1H, |
| | | | d, 8.0Hz), 6.98(1H, t, 7.8Hz), 6.43-6.39(3H, |
| | | | m), 3.92(2H, s). |
| I-32 | 1.70 | 324.00 | 11.03(1H, s), 10.34(1H, s), 7.97(1H, d, |
| | | | 1.5Hz), 7.70(1H, dd, 2.0Hz, 8.3Hz), 7.57(1H, |
| | | | d, 8.0Hz), 6.65(1H, d, 8.0Hz), 6.23(1H, |
| | | | d, 2.3Hz), 6.13(1H, dd, 2.3Hz, |
| | | | 8.0Hz), 3.68(2H, s). |
| I-33 | 1.82 | 346.00 | 11.00(1H, s), 10.46(1H, s), 7.97-7.95(2H, |
| | | | m), 7.70(1H, dd, 1.8Hz, 8.3Hz), 7.57-7.54(2H, |
| | | | m), 7.26-7.21(2H, m), 7.04(1H, t, |
| | | | 7.5Hz), 6.93(1H, d, 8.0Hz), 6.17(1H, d, |
| | | | 7.0Hz), 3.91(2H, d, 4.0Hz). |
| I-34 | 1.78 | 324.00 | 11.22(1H, s), 10.53(1H, s), 8.16(1H, d, |
| | | | 1.5Hz), 7.90(1H, dd, 8.3Hz, 1.8Hz), 7.77(1H, |
| | | | d, 8.1Hz), 6.83(1H, s), 6.80(1H, d, |
| | | | 8.1Hz), 6.30(1H, d, 8.1Hz), 3.94(2H, s), |
| | | | 2.14(3H, s), 2.12(3H, s) |
| I-35 | 1.66 | 356.00 | 11.23(1H, s), 10.60(1H, s), 8.16(1H, d, |
| | | | 1.8Hz), 7.89(1H, dd, 8.3Hz, 2.0Hz), 7.77(1H, |
| | | | d, 8.3Hz), 6.07(1H, t, 6.1Hz), 5.79-5.78(3H, |
| | | | m), 3.96(2H, d, 6.1Hz), 3.64(6H, |
| | | | s). |
| I-36 | 1.71 | 359.00 | 11.23(1H, s), 10.59(1H, s), 8.16(1H, d, |
| | | | 1.5Hz), 7.89(1H, dd, 8.1Hz, 1.8Hz), 7.77(1H, |
| | | | d, 8.1Hz), 6.94(1H, d, 8.8Hz), 6.71(1H, |
| | | | d, 2.8Hz), 6.53(1H, dd, 8.8Hz, 2.8Hz), |
| | | | 3.90(2H, s), 3.70(3H, s). |
| I-37 | 1.71 | 313.00 | 11.23(1H, s), 10.64(1H, s), 8.16(1H, d, |
| | | | 1.8Hz), 7.89(1H, dd, 8.1Hz, 1.8Hz), 7.77(1H, |
| | | | d, 8.1Hz), 7.09(1H, q, 8.1Hz), 6.44-6.32(3H, |
| | | | m), 3.96(2H, d, 6.0Hz). |
| I-38 | 1.76 | 310.00 | 11.29(1H, s), 10.57(1H, s), 8.17(1H, d, |
| | | | 1.5Hz), 7.91(1H, dd, 8.1Hz, 1.5Hz), 7.77(1H, |
| | | | d, 8.1Hz), 7.01-6.98(2H, m), 6.55(1H, |
| | | | t, 7.3Hz), 6.40(1H, d, 7.8Hz), 3.98(2H, |
| | | | s), 2.16(3H, s) |
| I-39 | 1.77 | 348.00 | 11.23(1H, s), 10.62(1H, s), 8.15(1H, d, |
| | | | 1.3Hz), 7.89(1H, dd, 8.1Hz, 1.3Hz), 7.78(1H, |
| | | | d, 8.1Hz), 7.16-7.11(1H, m), 6.74-6.71(1H, |
| | | | m), 6.58-6.54(1H, m), 6.31-6.28(1H, |
| | | | m), 3.95(2H, d, 6.2Hz). |
| I-40 | 1.75 | 327.00 | 11.20(bs), 10.64(bs), 8.19(1H, d, 1.6Hz), |
| | | | 8.03(1H, dd, 8.2Hz, 1.8Hz), 7.86(1H, d, |
| | | | 8.2Hz), 7.29-7.25(1H, m), 6.85-645(3H, |
| | | | m), 4.76(2H, s) 3.79(3H, s). |
| I-41 | 1.36 | 347.00 | 11.25(1H, s), 10.82(1H, s), 8.10(1H, d, |
| | | | 1.2Hz), 7.82(1H, dd, 1.8Hz, 8.4Hz), 7.78(1H, |
| | | | d, 8.1Hz), 7.45-7.39(4H, m), 3.95(2H, |
| | | | s). |
| I-42 | 1.25 | 331.00 | 11.19(1, s), 10.76(1H, s), 8.03(1H, d, |
| | | | 1.3Hz), 7.74(1H, dd, 1.8Hz, 8.3Hz), 7.72(1H, |
| | | | t, 8.1Hz), 7.46(1H, dt, 1.5Hz, 7.6Hz), |
| | | | 7.29-7.12(3H, m), 3.86(1H, s). |
| I-43 | 1.37 | 359.00 | 11.24(1H, s), 10.78(1H, s), 8.10(1H, d, |
| | | | 1.3Hz), 7.82(1H, dd, 1.8Hz, 8.3Hz), 7.79(1H, |
| | | | t, 8.1Hz), 7.37(2H, dd, 1.8Hz, 8.3Hz), |
| | | | 7.22(2H, dd, 2.0Hz, 8.6Hz), 3.87(2H, s). |
| I-44 | 1.46 | 381.00 | 11.26(1H, s), 10.95(1H, s), 8.11(1H, d, |
| | | | 1.6Hz), 7.83(1H, dd, 1.6Hz, 8.2Hz), 7.79(1H, |
| | | | t, 8.2Hz), 7.66(1H, d, 2.2Hz), 7.49(1H, |
| | | | t, 8.5Hz), 7.45(1H, dd, 2.2Hz, 8.5Hz), |
| | | | 4.06(2H, s). |
| I-45 | 1.33 | 295.00 | 11.00(1H, s), 8.39(1H, d, 1.8Hz), 8.11(1H, |
| | | | dd, 8.1Hz, 1.8Hz), 8.01(1H, d, 8.3Hz), |
| | | | 7.51-7.46(1H, m), 7.44-7.36(3H, m), |
| | | | 4.00(2H, s), 2.53(3H, s) |
| I-46 | 1.28 | 311.00 | 10.68(1H, s), 8.15(1H, d, 1.5Hz), 7.88(1H, |
| | | | dd, 8.3Hz, 1.8Hz), 7.77(1H, d, |
| | | | 8.1Hz), 7.30-7.21(2H, m), 6.99(1H, d, |
| | | | 7.6Hz), 6.92(1H, dt, 7.3Hz, 1.0Hz), 3.76(3H, |
| | | | s), 3.70(2H, s) |
| I-47 | 1.39 | 359.00 | 10.93(1H, s), 8.28(1H, d, 1.8Hz), 8.00(1H, |
| | | | dd, 8.1Hz, 1.8Hz), 7.91(1H, d, |
| | | | 8.3Hz), 7.71(1H, s), 7.63-7.59(1H, m), |
| | | | 7.51-7.42(2H, m), 3.89(2H, s) |
| I-48 | 1.40 | 359.00 | 10.60(1H, s), 7.95(1H, d, 1.8Hz), 7.67(1H, |
| | | | dd, 8.3Hz, 1.8Hz), 7.58(1H, d, |
| | | | 8.1Hz), 7.35(2H, d, 8.3Hz), 7.12(2H, d, |
| | | | 8.3Hz), 3.53(2H, s) |
| I-49 | 1.26 | 311.00 | 10.76(1H, s), 8.16(1H, d, 1.5Hz), 7.88(1H, |
| | | | dd, 8.1Hz, 1.8Hz), 7.78(1H, d, |
| | | | 8.1Hz), 7.25(1H, t, 8.3Hz), 6.94-6.90(2H, |
| | | | m), 6.87-6.82(1H, m), 3.75(3H, s), 3.69(2H, |
| | | | s) |
| I-50 | 1.03 | 340.00 | 11.22(1H, s), 10.56(1H, s), 8.17(1H, d, |
| | | | 1.3Hz), 7.90(1H, dd, 8.3Hz, 1.8Hz), 7.77(1H, |
| | | | d, 8.3Hz), 6.67(1H, d, 8.3Hz), 6.34(1H, |
| | | | d, 2.3Hz), 6.00(1H, dd, 8.3Hz, |
| | | | 2.3Hz), 5.84(2H, s), 3.87(2H, s) |
| I-51 | 1.14 | 310.00 | 11.22(1H, s), 10.57(1H, s), 8.16(1H, d, |
| | | | 1.5Hz), 7.90(1H, dd, 8.1Hz, 1.8Hz), 7.76(1H, |
| | | | d, 8.3Hz), 6.92(2H, m), 6.53(2H, |
| | | | m), 3.90(2H, s), 2.14(3H, s). |
| I-52 | 1.27 | 330.00 | 11.23(1H, s), 10.61(1H, s), 8.16(1H, d, |
| | | | 1.3Hz), 7.89(1H, dd, 8.1Hz, 1.8Hz), 7.77(1H, |
| | | | d, 8.3Hz), 7.11(2H, m), 6.60(2H, |
| | | | m), 3.94(2H, s). |
| I-53 | 1.12 | 296.00 | 11.22(1H, s), 10.60(1H, s), 8.17(1H, d, |
| | | | 1.5Hz), 7.90(1H, dd, 8.4Hz, 2.0Hz), 7.77(1H, |
| | | | d, 8.3Hz), 7.09(2H, m), 6.61-6.57(3H, |
| | | | m), 3.93(2H, s). |
| I-54 | 1.37 | 365.00 | 11.27(1H, s), 10.90(1H, s), 8.12(1H, d, |
| | | | 1.3Hz), 7.84(1H, dd, 1.8Hz, 8.4Hz), 7.80(1H, |
| | | | d, 8.2Hz), 7.60-7.55(2H, m), 7.30(1H, |
| | | | dt, 2.8Hz, 8.6Hz), 3.99(2H, s). |
| I-55 | 3.91 | 391.00 | 11.26(1H, s), 10.85(1H, s), 8.10(1H, d, |
| | | | 1.5Hz), 7.83(1H, dd, 1.8Hz, 8.1Hz), 7.78(1H, |
| | | | d, 8.3Hz), 7.63(1H, dd, 1.8Hz, |
| | | | 1.8Hz), 7.41(1H, d, 1.5Hz), 7.39(1H, d, |
| | | | 1.8Hz), 7.28(1H, d, 1.0Hz), 4.00(2H, s). |
| I-56 | 1.40 | 363.00 | 11.23(1H, s), 10.87(1H, s), 8.11(1H, d, |
| | | | 1.5Hz), 7.94-7.76(6H, m), 7.55-7.45(3H, |
| | | | m), 4.02(2H, s) |
| I-57 | 1.28 | 349.00 | 11.42(1H, br s), 10.87(1H, s), 8.18(1H, d, |
| | | | 1.3Hz), 7.89(1H, d, 1.5Hz), 7.75-7.69(1H, |
| | | | m), 7.47(1H, dt, .2.8Hz, 9.6Hz), 7.24(1H, |
| | | | ddt, 8.6Hz, 1.0Hz, .2.8Hz), 3.95(2H, s). |
| I-58 | 1.33 | 347.00 | 11.31(1H, s), 10.96(1H, s), 8.16(1H, d, |
| | | | 1.5Hz), 7.89(1H, dd, 1.8Hz, 8.1Hz), 7.83(1H, |
| | | | d, 8.1Hz), 7.54(1H, s), 7.52(1H, s), |
| | | | 7.40(1H, dt, 1.0Hz, 7.3Hz), 7.27(1H, dt, |
| | | | 1.2Hz, 7.3Hz), 4.08(2H, s) |
| I-59 | 1.39 | 341.00 | 11.25(1H, s), 10.78(1H, s), 8.10(1H, d, |
| | | | 1.3Hz), 7.82(1H, dd, 1.8Hz, 8.1Hz), 7.77(1H, |
| | | | d, 8.1Hz), 7.03(2H, s), 6.84(1H, s), |
| | | | 3.89(2H, s), 2.22(6H, s) |
| I-60 | 1.34 | 365.00 | 11.25(1H, s), 10.81(1H, s), 8.09(1H, d, |
| | | | 1.2Hz), 7.80(1H, d, 1.7Hz), 7.78(1H, dd, |
| | | | 1.1Hz, 8.1Hz), 7.70(1H, dd, 2.0Hz, 6.9Hz), |
| | | | 7.46-7.38(2H, m), 3.96(2H, s). |
| I-61 | 1.37 | 341.00 | 11.09(1H, s), 10.65(1H, s), 7.94(1H, d, |
| | | | 1.3Hz), 7.66(1H, dd, 1.5Hz, 8.1Hz), 7.62(1H, |
| | | | d, 8.1Hz), 7.07(1H, s), 7.94(1H, d, |
| | | | 7.6Hz), 6.78(1H, d, 7.3Hz), 3.72(2H, s), |
| | | | 2.12(3H, s), 2.07(3H, s). |
| I-62 | 1.38 | 341.00 | 11.24(1H, s), 10.62(1H, s), 8.04(1H, d, |
| | | | 0.8Hz), 7.76(1H, d, 8.0Hz), 7.73(1H, dd, |
| | | | 1.6Hz, 8.2Hz), 7.19-7.11(3H, m), 3.50(2H, |
| | | | s), 2.47(6H, s). |
| I-63 | 1.04 | 345.00 | 11.29(1H, s), 10.94(1H, s), 8.03(1H, d, |
| | | | 1.5Hz), 7.94-7.92(2H, m), 7.81-7.66(5H, |
| | | | m), 4.55(2H, s). |
| I-64 | 1.44 | 381.00 | 11.17(1H, s), 10.77(1H, s), 8.00(1H, d, |
| | | | 1.5Hz), 7.73(1H, dd, 1.8Hz, 8.3Hz), 7.69(1H, |
| | | | d, 8.1Hz), 7.62(1H, d, 2.2Hz), 7.49(1H, |
| | | | d, 8.3Hz), 7.29(1H, dd, 2.3Hz, |
| | | | 8.4Hz), 3.93(2H, s). |
| I-65 | 1.31 | 327.00 | 11.37(1H, s), 10.91(1H, s), 8.23(1H, d, |
| | | | 1.5Hz), 7.94(1H, dd, 1.8Hz, 8.3Hz), 7.89(1H, |
| | | | d, 8.3Hz), 7.36-7.32(3H, m), 7.14(1H, |
| | | | d, 6.3Hz), 4.03(2H, s), 2.39(3H, s). |
| I-66 | 1.39 | 381.00 | 11.43(1H, s), 11.05(1H, s), 8.26(1H, d, |
| | | | 1.5Hz), 7.99(1H, dd, 1.8Hz, 8.1Hz), 7.96(1H, |
| | | | d, 8.1Hz), 7.93(1H, s), 7.90-7.83(1H, |
| | | | m), 7.73(1H, d, 5.3Hz), 4.24(2H, s), |
| I-67 | 1.41 | 397.00 | 11.43(1H, s), 11.01(1H, s), 8.28(1H, d, |
| | | | 1.8Hz), 7.99(1H, dd, 1.5Hz, 8.4Hz), 7.95(1H, |
| | | | d, 8.4Hz), 7.69(2H, d, 8.6Hz), 7.51(2H, |
| | | | d, 8.8Hz), 4.15(2H, s), |
| I-68 | 1.85 | 349.00 | 11.41(1H, s), 11.01(1H, s), 8.31(1H, d, |
| | | | 1.8Hz), 8.04(1H, dd, 8.1Hz, 1.8Hz), 7.95(1H, |
| | | | d, 8.3Hz), 7.89(1H, s), 7.84-7.79(2H, |
| | | | m), 7.58(1H, t, 7.6Hz), 4.05(2H, s) |
| I-69 | 1.76 | 359.00 | 10.69(1H, s), 8.06(1H, d, 1.8Hz), 7.80(1H, |
| | | | dd, 8.3Hz, 1.8Hz), 7.70(1H, d, |
| | | | 8.3Hz), 7.03(1H, s), 6.97(1H, s), 6.01(2H, |
| | | | s), 3.74(2H, s) |
| I-70 | 1.73 | 419.00 | 10.92(1H, s), 8.31(1H, d, 1.3Hz), 8.04(1H, |
| | | | dd, 8.3Hz, 1.8Hz), 7.94(1H, d, |
| | | | 8.3Hz), 7.31(1H, s), 7.24(1H, s), 4.01(2H, |
| | | | s), 3.94(3H, s), 3.92(3H, s) |
| I-71 | 1.88 | 350.00 | 11.22(bs), 10.56(1H, s), 8.16(1H, d, |
| | | | 1.5Hz), 7.92(1H, dd, 8.3Hz, 2.0Hz), 7.77(1H, |
| | | | d, 8.1Hz), 6.88(1H, t, 7.6Hz), 6.37(1H, |
| | | | d, 7.6Hz), 6.21(1H, d, 7.8Hz), 5.28(1H, |
| | | | t, 5.8Hz), 2.66-2.63(2H, m), 1.81-1.77(2H, |
| | | | m), 1.75-1.64(2H, m), 1.18-1.149(2H, |
| I-72 | 1.01 | 335.00 | 10.86(1H, s), 10.61(1H, s), 8.18(1H, d, |
| | | | 1.8Hz), 7.92(1H, dd, 8.3Hz, 2.0Hz), 7.76(1H, |
| | | | d, 8.1Hz), 7.13(1H, t, 2.8Hz), 6.83(1H, |
| | | | t, 7.38Hz), 6.71(1H, d, 8.1Hz), 6.60-6.59(1H, |
| | | | m), 6.07(1H, t, 6.3Hz), 5.96(1H, |
| | | | d, 7.6Hz), 4.04(2H, d, 6.1Hz). |
| I-73 | 1.32 | 331.00 | 11.26(1H, s), 10.69(1H, s), 8.17(1H, d, |
| | | | 1.7Hz), 7.95(1H, dd, 8.1Hz, 1.7Hz), 7.79(1H, |
| | | | d, 8.2Hz), 7.34(1H, t, 8.2Hz), 7.12(1H, |
| | | | t, 2.1Hz), 7.04(1H, dd, 8.0Hz, 1.8Hz), |
| | | | 7.00(1H, dd, 8.3Hz, 2.4Hz), 4.83(2H, s). |
| I-74 | 1.84 | 331.00 | 11.25(1H, s), 10.69(1H, s), 8.17(1H, d, |
| | | | 1.6Hz), 7.94(1H, dd, 8.2Hz, 1.8Hz), 7.78(1H, |
| | | | d, 8.2Hz), 7.36-7.34(2H, m), 7.05-7.03(2H, |
| | | | m), 4.78(2H, s). |
| I-75 | 1.49 | 347.00 | 10.82(bs), 10.72(bs), 8.26-8.23(1H, m), |
| | | | 7.90-7.88(1H, m), 7.64(1H, d, 8.3Hz), |
| | | | 7.56-7.49(2H, m), 7.38(1H, t, 7.8Hz), |
| | | | 6.99-6.94(2H, m), 6.79(2H, s), 5.47(2H, |
| | | | s). |
| I-76 | 1.90 | 325.00 | 11.25(1H, s), 10.67(1H, s), 8.17(1H, d, |
| | | | 1.76Hz), 7.93(1H, dd, 8.3Hz, 1.76), 7.79(1H, |
| | | | d, 8.1Hz), 7.03(1H, t, 7.8Hz), 6.80(1H, |
| | | | d, 7.6Hz), 6.73(1H, d, 8.3Hz), 4.76(2H, |
| | | | s). |
| I-77 | 1.40 | 341.00 | 11.34(1H, s), 10.86(1H, s), 8.19(1H, d, |
| | | | 1.2Hz), 7.91(1H, dd, 1.8Hz, 8.3Hz), 7.87(1H, |
| | | | d, 8.1Hz), 7.39(1H, d, 8.1Hz), 7.15(1H, |
| | | | s), 7.09(1H, d, 7.8Hz), 3.90(2H, s), |
| | | | 2.40(3H, s), 2.33(3H, s). |
| I-78 | 1.38 | 381.00 | 11.11(1H, s), 10.73(1H, s), 7.95(1H, d, |
| | | | 1.3Hz), 7.67(1H, dd, 1.5Hz, 8.3Hz), 7.64(1H, |
| | | | d, 8.1Hz), 7.61-7.58(2H, m), 7.51(1H, |
| | | | t, 7.8Hz), 7.28(1H, t, 7.6Hz), 3.91(2H, |
| | | | s). |
| I-79 | 1.41 | 381.00 | 11.19(1H, s), 10.87(1H, s), 8.04(1H, d, |
| | | | 1.5Hz), 7.77(1H, dd, 1.8Hz, 8.1Hz), 7.72(1H, |
| | | | d, 8.1Hz), 7.40(1H, dd, 1.5Hz, |
| | | | 7.8Hz), 7.37(1H, dd, 1.5Hz, 8.1Hz), 7.30(1H, |
| | | | t, 8.1Hz), 4.03(2H, s). |
| I-80 | 1.96 | 381.00 | 11.22(1H, s), 10.67(1H, s), 8.06(1H, d, |
| | | | 1.5Hz), 7.79(1H, dd, 1.8Hz, 8.1Hz), 7.74(1H, |
| | | | d, 8.1Hz), 7.39(1H, t, 1.8Hz), 4.04(2H, |
| | | | s). |
| I-81 | 1.63 | 329.00 | 11.41(1H, s), 10.89(1H, s), 9.76(1H, s), |
| | | | 8.28(1H, d, 1.5Hz), 7.99(1H, dd, 1.8Hz, |
| | | | 8.3Hz), 7.94(1H, d, 8.1Hz), 7.28(1H, t, |
| | | | 7.8Hz), 6.97-6.94(2H, m), 6.76(1H, ddd, |
| | | | 0.8Hz, 2.3Hz, 8.1Hz), 4.04(2H, s). |
| I-82 | 1.40 | 397.00 | 11.25(1H, s), 10.89(1H, s), 8.10(1H, d, |
| | | | 1.3Hz), 7.82(1H, dd, 1.8Hz, 8.1Hz), 7.78(1H, |
| | | | d, 8.1Hz), 7.60-7.58(1H, m), 7.43-7.32(3H, |
| | | | m), 4.01(2H, s). |
| I-83 | 1.15 | 281.00 | 11.22(1H, s), 10.77(1H, s), 8.14(1H, d, |
| | | | 1.0Hz), 7.86(1H, dd, 8.1Hz, 1.0Hz), 7.75(1H, |
| | | | d, 8.1Hz), 7.33-7.24(5H, m), 3.71(2H, |
| | | | s). |
| I-84 | 1.29 | 311.00 | 10.64(1H, s), 8.19(1H, d, 1.8Hz), 7.96(1H, |
| | | | dd, 8.4Hz, 1.8Hz), 7.79(1H d, |
| | | | 8.4Hz), 7.12-7.10(2H, m), 6.91-6.89(2H, |
| | | | m), 4.72(2H, s), 2.23(3H, s). |
| I-85 | 1.30 | 336.00 | 11.32(bs),, 10.57(1H, s), 8.17(1H, d, |
| | | | 1.5Hz), 7.91(1H, dd, 8.1Hz, 1.8Hz), 7.77(1H, |
| | | | d, 8.1Hz), 6.90(1H, t, 7.6Hz), 6.53(1H, |
| | | | d, 7.4Hz), 6.22(1H, d, 7.8Hz), 5.43(1H, |
| | | | t, 6.0Hz), 3.95(2H, d, 6.0Hz), 2.82-2.79(2H, |
| | | | m), 2.73-2.71(2H, m), 2.03-1.98(2H, |
| | | | m). |
| I-86 | 1.29 | 331.00 | 11.26(bs), 10.78(1H, s), 8.15(1H, d, |
| | | | 1.8Hz), 7.89(1H, dd, 8.1Hz, 1.9Hz), 7.79(1H, |
| | | | d, 8.1Hz), 7.46(1H, dd, 7.8Hz, |
| | | | 1.6Hz), 7.32-7.27(1H, m), 7.09(1H, dd, |
| | | | 8.3Hz, 1.3Hz), 7.00(1H, dt, 7.8Hz, 1.5Hz), |
| | | | 4.91(2H, s). |
| I-87 | 1.19 | 327.00 | 11.25(bs), 10.63(1H, s), 8.19(1H, d, |
| | | | 1.8Hz), 7.97(1H, dd, 8.1Hz, 1.8Hz), 7.79(1H, |
| | | | d, 8.1Hz), 6.97-6.87(4H, m), 4.69(2H, |
| | | | s), 3.69(3H, s). |
| I-88 | 1.13 | 326.00 | 10.60(bs), 8.16(1H, d, 1.8Hz), 7.90(1H, |
| | | | dd, 8.1Hz, 1.8Hz), 7.77(1H, d, 8.1Hz), 6.99(1H, |
| | | | t, 7.8Hz), 6.21-6.15(3H, m), 6.07(1H, |
| | | | t, 6.0Hz), 3.91(2H, d, 6.1Hz), 3.65(3H, s). |
| I-89 | 1.40 | 397.00 | 11.30(1H, s), 10.91(1H, s), 8.14(1H, d, |
| | | | 1.1Hz), 7.86(1H, dd, 1.5Hz, 8.3Hz), 7.82(1H, |
| | | | d, 8.1Hz), 7.52-7.45(3H, m), 7.23(1H, |
| | | | d, 7.8Hz), 4.07(2H, s). |
| I-90 | 1.18 | 299.00 | 11.23(1H, s), 10.77(1H, s), 8.15(1H, d, |
| | | | 1.8Hz), 7.86(1H, dd, 1.8Hz, 8.4Hz), 7.77(1H, |
| | | | d, 8.1Hz), 7.39-7.35(2H, m), 7.19-7.14(2H, |
| | | | m), 3.73(2H, s). |
| I-91 | 1.23 | 333.00 | 11.24(1H, s), 10.94(1H, s), 8.12(1H, d, |
| | | | 1.5Hz), 7.86(1H, dd, 1.8Hz, 8.1Hz), 7.78(1H, |
| | | | d, 8.1Hz), 7.42-7.36(2H, m), 7.29-7.25(1H, |
| | | | m), 3.97(2H, s). |
| I-92 | 1.21 | 317.00 | 10.02(2H br.s), 8.30(1H, d, 1.8Hz), 8.00(1H, |
| | | | dd, 1.8Hz, 8.1Hz), 7.81(1H, d, 8.1Hz), |
| | | | 7.56-7.50(1H, m), 7.09-7.01(2H, m), 3.90(2H, |
| | | | s). |
| I-93 | 1.19 | 317.00 | 10.07(1H, s), 10.01(1H, s), 8.27(1H, d, |
| | | | 1.8Hz), 7.98(1H, dd, 2.0Hz, 8.4Hz), 7.79(1H, |
| | | | d, 8.1Hz), 7.46-7.38(1H, m), 7.07-7.04(2H, |
| | | | m), 3.94(2H, s). |
| I-94 | 1.81 | 311.00 | 11.25(bs), 10.64(1H, s), 8.19(1H, d, |
| | | | 1.5Hz), 7.97(1H, dd, 8.1Hz, 1.8Hz), 7.79(1H, |
| | | | d, 8.3Hz), 7.19(1H, t, 7.8Hz), 6.84-6.79(3H, |
| | | | m), 4.74(2H, s), 2.28(3H, s). |
| I-95 | 1.74 | 295.00 | 11.21(bs), 10.51(1H, s), 8.15(1H, d, |
| | | | 1.5Hz), 7.82(1H, dd, 8.1Hz, 1.8Hz), 7.77(1H, |
| | | | d, 8.1Hz), 7.30-7.24(4H, m), 7.20-7.18(1H, |
| | | | m), 2.95-2.91(2H, m), 2.72-2.68(2H, |
| | | | m). |
| I-96 | 1.88 | 325.00 | 11.25(bs), 10.65(1H, s), 8.19(1H, d, |
| | | | 1.5Hz), 7.96(1H, dd, 8.1Hz, 1.8Hz), 7.79(1H, |
| | | | d, 8.3Hz), 7.15-7.13(2H, m), 6.93-6.91(2H, |
| | | | m), 4.73(2H, s), 2.53(2H, q, |
| | | | 7.6Hz), 1.14(2H, t, 7.5Hz). |
| I-97 | 1.26 | 311.00 | 11.24(bs), 10.70(1H, s), 8.17(1H, s), 7.96(1H, |
| | | | d, 8.1Hz), 7.76(1H, d, 8.1Hz), 7.31-7.27(2H, |
| | | | m), 6.94-6.93(3H, m), 4.92(1H, |
| | | | q, 6.5Hz), 1.58(3H, d, 6.5Hz). |
| I-98 | 1.19 | 341.00 | 11.20(bs), 10.60(1H, s), 8.14(1H, d, |
| | | | 1.8Hz), 7.87(1H, dd, 8.3Hz, 2.0Hz), 7.75(1H, |
| | | | d, 8.3Hz), 7.12(1H, d, 8.3Hz), 6.54(1H, |
| | | | d, 2.5Hz), 6.48(1H, dd, 8.1Hz, |
| | | | 2.3Hz), 3.75(3H, s), 3.74(3H, s), 3.60(2H, |
| | | | s). |
| I-99 | 1.28 | 330.00 | 11.23(bs), 10.63(1H, s), 8.16(1H, d, |
| | | | 1.5Hz), 7.89(1H, dd, 8.3Hz, 2.0Hz), 7.78(1H, |
| | | | d, 8.3Hz), 7.11-7.07(1H, m), 6.63-6.62(1H, |
| | | | m), 6.59-6.53(2H, m), 3.96(2H, |
| | | | s). |
| I-100 | 1.20 | 327.00 | 11.25(bs), 10.69(1H, s), 8.17(1H, d, |
| | | | 1.5Hz), 7.92(1H, dd, 8.3Hz, 1.8Hz), 7.78(1H, |
| | | | d, 8.1Hz), 7.03-6.94(3H, m), 6.89-6.85(1H, |
| | | | m), 4.74(2H, s), 3.80(3H, s). |
| I-101 | 1.78 | 335.00 | 11.19(1H, s), 10.81(1H, s), 8.07(1H, d, |
| | | | 1.8Hz), 7.81(1H, dd, 8.3Hz, 1.8Hz), 7.73(1H, |
| | | | d, 8.1Hz), 7.46-7.38(1H, m), 7.17-7.11(1H, |
| | | | m), 3.86(2H, s) |
| I-102 | 1.55 | 297.00 | 9.84(1H, br s), 9.72(1H, s), 8.18(1H, s), |
| | | | 8.16(1H, d, 2.0Hz), 7.82(1H, dd, 8.3Hz, |
| | | | 1.8Hz), 7.63(1H, d, 8.1Hz), 7.01(1H, t, 7.8Hz), |
| | | | 6.76(1H, s), 6.71(1H, d, 7.6Hz), 6.61(1H, |
| | | | dd, 8.3Hz, 2.3Hz), 3.56(2H, s) |
| I-103 | 1.77 | 325.00 | 11.08(1H, s), 10.60(1H, s), 8.01(1H, d, |
| | | | 1.5Hz), 7.72(1H, dd, 8.1Hz, 2.0Hz), 7.62(1H, |
| | | | d, 8.3Hz), 7.08(1H, t, 7.8Hz), 6.77-6.73(2H, |
| | | | m), 6.69-6.65(1H, m), 3.86(2H, |
| | | | q, 7.1Hz), 3.52(2H, s), 1.17(3H, t, 7.1Hz) |
| I-104 | 1.77 | 333.00 | 11.23(1H, s), 10.84(1H, s), 8.14(1H, d, |
| | | | 1.5Hz), 7.86(1H, dd, 1.8Hz, 8.3Hz), 7.78(1H, |
| | | | d, 8.1Hz), 7.52-7.44(2H, m), 7.23(1H, |
| | | | dt, 2.8Hz, 8.6Hz), 3.91(2H, s). |
| I-105 | 1.88 | 368.00 | 9.89(2H, br s), 8.12(1H, d, 1.5Hz), 7.83(1H, |
| | | | dd, 1.8Hz, 8.3Hz), 7.65(1H, d, |
| | | | 8.1Hz), 7.53(1H, d, 7.1Hz), 7.38(1H, d, |
| | | | 9.9Hz), 3.89(2H, s). |
| I-106 | 1.79 | 332.00 | 11.06(1H, s), 10.66(1H, s), 7.95(1H, d, |
| | | | 1.5Hz), 7.69(1H, dd, 1.5Hz, 8.1Hz), 7.60(1H, |
| | | | d, 8.4Hz), 7.35(1H, dd, 2.5Hz, |
| | | | 6.3Hz), 7.25-7.21(1H, m), 7.09(1H, t, |
| | | | 9.1Hz), 3.67(2H, s). |
| I-107 | 1.73 | 299.00 | 11.24(1H, s), 10.79(1H, s), 8.15(1H, d, |
| | | | 1.4Hz), 7.87(1H, dd, 2.0Hz, 8.3Hz), 7.77(1H, |
| | | | d, 8.1Hz), 7.41-7.36(1H, m), 7.20-7.17(2H, |
| | | | m), 7.13-7.08(1H, m), 3.77(2H, |
| | | | s). |
| I-108 | 1.85 | 348.00 | 11.24(1H, s), 10.79(1H, s), 8.13(1H, d, |
| | | | 1.5Hz), 7.86(1H, d, 1.8Hz, 8.3Hz), 7.77(1H, |
| | | | d, 8.1Hz), 7.62(1H, d, 1.8Hz), 7.60(1H, |
| | | | d, 8.4Hz), 7.34(1H, dd, 2.0Hz, |
| | | | 8.4Hz), 3.78(2H, s). |
| I-109 | 1.90 | 325.00 | 11.25(1H, s), 10.63(1H, s), 8.17(1H, d, |
| | | | 1.8Hz), 7.93(1H, dd, 8.1Hz, 1.8Hz), 7.79(1H, |
| | | | d, 8.3Hz), 6.98(1H, s), 6.92(1H, dd, |
| | | | 8.3Hz, 2.0Hz), 6.76(1H, d, 8.1Hz), 4.73(2H, |
| | | | s), 2.21(3H, s), 2.19(3H, s). |
| I-110 | 1.89 | 325.00 | 10.63(1H, s), 8.19(1H, d, 1.8Hz), 7.97(1H, |
| | | | dd, 8.1Hz, 1.8Hz), 7.78(1H, d, |
| | | | 8.3Hz), 7.04(1H, d, 8.3Hz), 6.82(1H, d, |
| | | | 2.5Hz), 6.71(1H, dd, 8.3Hz, 2.5Hz), 4.70(2H, |
| | | | s), 2.18(3H, s), 2.13(3H, s). |
| I-111 | 1.38 | 325.00 | 11.25(1H, s), 10.63(1H, s), 8.18(1H, d, |
| | | | 1.8Hz), 7.94(1H, dd, 8.1Hz, 1.8Hz), 7.79(1H, |
| | | | d, 8.1Hz), 7.04(1H, d, 7.6Hz), 6.72-6.69(2H, |
| | | | m), 4.76(2H, s), 2.24(3H, s), |
| | | | 2.20(3H, s). |
| I-112 | 1.26 | 315.00 | 11.23(1H, s), 10.78(1H, s), 8.14(1H, d, |
| | | | 1.5Hz), 7.86(1H, dd, 8.1Hz, 1.5Hz), 7.77(1H, |
| | | | d, 8.1Hz), 7.42-7.29(4H, m), 3.76(2H, |
| | | | s), |
| I-113 | 1.82 | 315.00 | 11.29(1H, s), 10.84(1H, s), 8.20(1H, d, |
| | | | 1.5Hz), 7.92(1H, dd, 2.0Hz, 8.3Hz), 7.83(1H, |
| | | | d, 8.1Hz), 7.47-7.41(4H, m), 3.79(2H, |
| | | | s). |
| I-114 | 1.20 | 317.00 | 11.24(1H, s), 10.84(1H, s), 8.13(1H, d, |
| | | | 1.5Hz), 7.86(1H, dd, 1.8Hz, 8.3Hz), 7.78(1H, |
| | | | d, 8.1Hz), 7.33-7.15(3H, m), 3.84(2H, |
| | | | s). |
| I-115 | 1.31 | 309.00 | 11.01(1H, s), 10.53(1H, s), 7.94(1H, d, |
| | | | 1.8Hz), 7.66(1H, dd, 1.8Hz, 8.4Hz), 7.55(1H, |
| | | | d, 8.1Hz), 6.86-6.84(2H, m), 6.77-6.75(1H, |
| | | | m), 3.50(2H, s), 2.03(3H, s), |
| | | | 2.02(3H, s). |
| I-116 | 1.32 | 367.00 | 11.17(1H, s), 10.76(1H, s), 8.05(1H, d, |
| | | | 1.8Hz), 7.78(1H, dd, 1.8Hz, 8.1Hz), 7.71(1H, |
| | | | d, 8.1Hz), 7.57-7.47(3H, m), 3.94(2H, |
| | | | s). |
| I-117 | 1.37 | 367.00 | 11.00(1H, s), 10.60(1H, s), 7.90(1H, d, |
| | | | 1.5Hz), 7.63(1H, dd, 1.8Hz, 8.1Hz), 7.54(1H, |
| | | | d, 8.1Hz), 7.37-7.35(2H, m), 7.33-7.30(1H, |
| | | | m), 3.69(2H, s). |
| I-118 | 1.78 | 315.00 | 11.25(1H, s), 10.66(1H, s), 8.18(1H, d, |
| | | | 1.7Hz), 7.96(1H, dd, 8.1Hz, 1.9Hz), 7.79(1H, |
| | | | d, 8.1Hz), 7.18-7.14(2H, m), 7.05-7.02(2H, |
| | | | m), 4.76(2H, s), |
| I-119 | 1.23 | 341.00 | 11.24(1H, s), 10.65(1H, s), 8.18(1H, d, |
| | | | 1.7Hz), 7.97(1H, dd, 8.3Hz, 1.9Hz), 7.77(1H, |
| | | | d, 8.1Hz), 6.94-6.92(2H, m), 6.87-6.85(2H, |
| | | | m), 4.81(1H, q, 6.6Hz), 1.54(3H, |
| | | | d, 6.6Hz). |
| I-121 | 1.90 | 383.00 | 9.88(2H, br s), 8.11(1H, d, 1.7Hz), 7.82(1H, |
| | | | dd, 2.0Hz, 8.3Hz), 7.65(1H, d, |
| | | | 8.1Hz), 7.63(1H, d, 7.9Hz), 7.56(1H, br s), |
| | | | 7.44(1H, d, 8.1Hz), 3.97(2H, s). |
| I-122 | 1.76 | 317.00 | 11.29(1H, s), 10.91(1H, s), 8.18(1H, d, |
| | | | 1.4Hz), 7.92(1H, dd, 1.7Hz, 8.3Hz), 7.83(1H, |
| | | | d, 8.3Hz), 7.45-7.38(1H, m), 7.31-7.22(2H, |
| | | | m), 3.95(2H, s). |
| I-123 | 1.84 | 367.00 | — |
| I-127 | 1.31 | 297.00 | 10.72(bs), 7.62(1H, d, 8.1Hz), 7.29-7.26(2H, |
| | | | m), 6.98-6.93(4H, m), 6.78(2H, bs), |
| | | | 5.24(2H, s). |

The activity of a compound utilized in this invention as an inhibitor of AKT, PDK1, p70S6K, or ROCK kinase may be assayed in vitro, in vivo or in a cell line according to methods known in the art. In vitro assays include assays that determine inhibition of either the phosphorylation activity or ATPase activity of activated AKT, PDK1, p70S6K, or ROCK. Alternate in vitro assays quantitate the ability of the inhibitor to bind to AKT, PDK1, p70S6K, or ROCK. Inhibitor binding may be measured by radiolabelling the inhibitor prior to binding, isolating the inhibitor/AKT, inhibitor/PDK1, inhibitor/p70S6K, or inhibitor/ROCK complex and determining the amount of radiolabel bound. Alternatively, inhibitor binding may be determined by running a competition experiment where compounds are incubated with AKT, PDK1, p70S6K, or ROCK bound to known radioligands. Detailed conditions for assaying a compound utilized in this invention as an inhibitor of AKT, PDK1, p70S6K, or ROCK kinase are set forth in the Examples below.

According to another embodiment, the invention provides a composition comprising a compound of this invention or a pharmaceutically acceptable derivative thereof and a pharmaceutically acceptable carrier, adjuvant, or vehicle. The amount of compound in the compositions of this invention is such that is effective to measurably inhibit a protein kinase, particularly AKT, PDK1, p70S6K, or ROCK kinase, in a biological sample or in a patient. Preferably the composition of this invention is formulated for administration to a patient in need of such composition. Most preferably, the composition of this invention is formulated for oral administration to a patient.

The term “patient”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The term “measurably inhibit”, as used herein means a measurable change in AKT, PDK1, p70S6K, or ROCK activity between a sample comprising said composition and a AKT, PDK1, p70S6K, or ROCK kinase and an equivalent sample comprising AKT, PDK1, p70S6K, or ROCK kinase in the absence of said composition.

A “pharmaceutically acceptable salt” means any non-toxic salt or salt of an ester of a compound of this invention that, upon administration to a recipient, is capable of providing, either directly or indirectly, a compound of this invention or an inhibitorily active metabolite or residue thereof. As used herein, the term “inhibitorily active metabolite or residue thereof” means that a metabolite or residue thereof is also an inhibitor of a AKT, PDK1, p70S6K, or ROCK family kinase.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g., sodium and potassium), alkaline earth metal (e.g., magnesium), ammonium and N⁺(C₁₋₄ alkyl)₄ salts. This invention also envisions the quaternization of any basic nitrogen-containing groups of the compounds disclosed herein. Water or oil-soluble or dispersible products may be obtained by such quaternization.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

The pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, the pharmaceutically acceptable compositions of this invention are formulated for oral administration.

The amount of the compounds of the present invention that may be combined with the carrier materials to produce a composition in a single dosage form will vary depending upon the host treated, the particular mode of administration. Preferably, the compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Depending upon the particular condition, or disease, to be treated or prevented, additional therapeutic agents, which are normally administered to treat or prevent that condition, may also be present in the compositions of this invention. As used herein, additional therapeutic agents that are normally administered to treat or prevent a particular disease, or condition, are known as “appropriate for the disease, or condition, being treated”.

For example, chemotherapeutic agents or other anti-proliferative agents may be combined with the compounds of this invention to treat proliferative diseases and cancer. Examples of known chemotherapeutic agents include, but are not limited to, Gleevec™, adriamycin, dexamethasone, vincristine, cyclophosphamide, fluorouracil, topotecan, taxol, interferons, and platinum derivatives.

Other examples of agents the inhibitors of this invention may also be combined with include, without limitation: treatments for Alzheimer's Disease such as Aricept® and Excelon®; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; and agents for treating immunodeficiency disorders such as gamma globulin.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

According to another embodiment, the invention relates to a method of inhibiting AKT, PDK1, p70S6K, or ROCK kinase activity in a biological sample comprising the step of contacting said biological sample with a compound of this invention, or a composition comprising said compound. Preferably, the method comprises the step of contacting said biological sample with a preferred compound of the present invention, as described herein supra.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of AKT, PDK1, p70S6K, or ROCK kinase activity in a biological sample is useful for a variety of purposes that are known to one of skill in the art. Examples of such purposes include, but are not limited to, blood transfusion, organ-transplantation, biological specimen storage, and biological assays.

Another aspect of this invention relates to a method for treating an AKT-, PDK1-, p70S6K-, or ROCK-mediated disease in a patient, which method comprises administering to a patient in need thereof, a therapeutically effective amount of a compound of the present invention, or a pharmaceutically acceptable composition comprising said compound. According to another embodiment, the invention relates to administering a compound of formula Ia or a pharmaceutically acceptable composition comprising said compound. Yet another embodiment relates to administering a compound of formula Ib, as described herein supra, or a pharmaceutically acceptable composition comprising said compound.

According to another embodiment, the present invention relates to a method for inhibiting AKT, PDK1, p70S6K, or ROCK in a patient, which method comprises administering to a patient in need thereof a compound of formula I, Ia, or Ib, or a pharmaceutically acceptable composition comprising said compound.

According to another embodiment, the invention provides a method for treating or lessening the severity of an AKT-mediated disease or condition in a patient comprising the step of administering to said patient a composition according to the present invention.

The term “AKT-mediated condition” or “disease”, as used herein, means any disease or other deleterious condition in which AKT is known to play a role. The term “AKT-mediated condition” or “disease” also means those diseases or conditions that are alleviated by treatment with a AKT inhibitor. Accordingly, another embodiment of the present invention relates to treating or lessening the severity of one or more diseases in which AKT is known to play a role. Specifically, the present invention relates to a method of treating or lessening the severity of a disease or condition selected from proliferative disorders, cancer, cardiovascular disorders, rheumatoid arthritis, and neurodegenerative disorders, wherein said method comprises administering a patient in need thereof a composition according to the present invention. According to another embodiment, said cancer is selected from pancreatic, prostate, or ovarian cancer.

According to another embodiment, the invention provides a method for treating or lessening the severity of an PDK1-mediated disease or condition in a patient comprising the step of administering to said patient a composition according to the present invention.

The term “PDK1-mediated condition” or “disease”, as used herein, means any disease or other deleterious condition in which PDK1 is known to play a role. The term “PDK1-mediated condition” or “disease” also means those diseases or conditions that are alleviated by treatment with a PDK1 inhibitor. Accordingly, another embodiment of the present invention relates to treating or lessening the severity of one or more diseases in which PDK1 is known to play a role. Specifically, the present invention relates to a method of treating or lessening the severity of a disease or condition selected from proliferative disorders, and pancreatic, prostate, or ovarian cancer, wherein said method comprises administering a patient in need thereof a composition according to the present invention.

According to another embodiment, the invention provides a method for treating or lessening the severity of a p70S6K-mediated disease or condition in a patient comprising the step of administering to said patient a composition according to the present invention.

The term “p70S6K-mediated condition” or “disease”, as used herein, means any disease or other deleterious condition in which p70S6K is known to play a role. The term “p70S6K-mediated condition” or “disease” also means those diseases or conditions that are alleviated by treatment with a p70S6K inhibitor. Accordingly, another embodiment of the present invention relates to treating or lessening the severity of one or more diseases in which p70S6K is known to play a role. Specifically, the present invention relates to a method of treating or lessening the severity of a disease or condition selected from proliferative disorders, such as cancer and tuberous sclerosis, wherein said method comprises administering a patient in need thereof a composition according to the present invention.

According to another embodiment, the invention provides a method for treating or lessening the severity of a ROCK-mediated disease or condition in a patient comprising the step of administering to said patient a composition according to the present invention.

The term “ROCK-mediated condition” or “disease”, as used herein, means any disease or other deleterious condition in which ROCK is known to play a role. The term “ROCK-mediated condition” or “disease” also means those diseases or conditions that are alleviated by treatment with a ROCK inhibitor. Accordingly, another embodiment of the present invention relates to treating or lessening the severity of one or more diseases in which ROCK is known to play a role. Specifically, the present invention relates to a method of treating or lessening the severity of a disease or condition selected from hypertension, angina pectoris, cerebrovascular contraction, asthma, ischemis/reperfusion, peripheral circulation disorder, premature birth, cancer, arteriosclerosis, spasm, retinopathy, inflammatory disorders, autoimmune disorders, AIDS, and osteoporosis, wherein said method comprises administering to a patient in need thereof a composition according to the present invention. The present invention also relates to a method of treating or lessening the severity of a disease or condition selected from hypertension, cerebral vasospasm, coronary vasospasm, bronchial asthma, preterm labor, erectile dysfunction, glaucoma, vascular smooth muscle cell proliferation, myocardial hypertrophy, malignoma, ischemia/reperfusion-induced injury, endothelial dysfunction, Crohn's Disease and colitis, neurite outgrowth, Raynaud's Disease, angina, Alzheimer's disease, benign prostatic hyperplasia, and atherosclerosis, wherein said method comprises administering to a patient in need thereof a composition according to the present invention.

According to another embodiment, the present invention relates to a method for treating or lessening the severity of a disease or condition selected from a proliferative disorder, a cardiac disorder, an inflammatory disorder, an autoimmune disorder, a viral disease, or a bone disorder, wherein said method comprises the step of administering an effective amount of a compound of the present invention.

According to another embodiment, the present invention relates to a method for treating or lessening the severity of a disease or condition selected from cancer and to a method for treating or lessening the severity of a disease or condition selected from rheumatoid arthritis, asthma, HIV, angina pectoris, peripheral circulation disorder, ischemia/reperfusion, hypertension, arteriosclerosis, or osteoporosis.

Preferably, the present invention relates to a method for treating or lessening the severity of a cancer.

More preferably, the present invention relates to a method for treating or lessening the severity of a cancer selected from brain (gliomas), breast, colon, head and neck, kidney, lung, liver, melanoma, ovarian, pancreatic, prostate, sarcoma, or thyroid.

Most preferably, the present invention relates to a method for treating or lessening the severity of pancreatic, prostate, or ovarian cancer.

In another most preferred embodiment, the present invention relates to a method for treating or lessening the severity of benign prostatic hyperplasia.

In an alternate embodiment, the methods of this invention that utilize compositions that do not contain an additional therapeutic agent, comprise the additional step of separately administering to said patient an additional therapeutic agent. When these additional therapeutic agents are administered separately they may be administered to the patient prior to, sequentially with or following administration of the compositions of this invention.

The compounds of this invention or pharmaceutical compositions thereof may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a compound of this invention. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccarides, polyethylene glycol, phospholipids or combinations thereof to impart controlled release characteristics in the composition. Implantable devices coated with a compound of this invention are another embodiment of the present invention.

In order that the invention described herein may be more fully understood, the following examples are set forth. It should be understood that these examples are for illustrative purposes only and are not to be construed as limiting this invention in any manner.

### EXAMPLES

### Example 1

### AKT-3 Inhibition Assay

Compounds were screened for their ability to inhibit AKT using a standard coupled enzyme assay (Fox et al., Protein Sci., (1998) 7, 2249). Assays were carried out in a mixture of 100 mM HEPES 7.5, 10 mM MgCl2, 25 mM NaCl, 1 mM DTT and 3% DMSO. Final substrate concentrations in the assay were 170 μM ATP (Sigma Chemicals) and 200 μM peptide (American Peptide, Sunnyvale, Calif.). Assays were carried out at 30° C. and 45 nM AKT. Final concentrations of the components of the coupled enzyme system were 2.5 mM phosphoenolpyruvate, 300 μM NADH, 30 μg/ML pyruvate kinase and 10 μg/ml lactate dehydrogenase.

An assay stock buffer solution was prepared containing all of the reagents listed above, with the exception of AKT, DTT, and the test compound of interest. 55 μl of the stock solution was placed in a 96 well plate followed by addition of 2 μl of 1 mM DMSO stock containing the test compound (final compound concentration 30 82 M). The plate was pre-incubated for about 10 minutes at 30° C. and the reaction initiated by addition of 10 μl of enzyme (final concentration 45 nM) and 1 mM DTT. Rates of reaction were obtained using a Molecular Devices SpectraMax Plus plate reader over a 15 minute read time at 30° C. Compounds showing greater than 50% inhibition versus standard wells containing the assay mixture and DMSO without test compound were titrated to determine IC₅₀ values.

### Example 2

### PDK-1 Inhibition Assay

Compounds were screened for their ability to inhibit PDK-1 using a radioactive-phosphate incorporation assay (Pitt and Lee, J. Biomol. Screen., (1996) 1, 47). Assays were carried out in a mixture of 100 mM HEPES (pH 7.5), 10 mM MgCl₂, 25 mM NaCl , 2 mM DTT. Final substrate concentrations in the assay were 40 μM ATP (Sigma Chemicals) and 65 μM peptide (PDKtide, Upstate, Lake Placid, N.Y.). Assays were carried out at 30° C. and 25 nM PDK-1 in the presence of ˜27.5 nCi/μL of [γ-³²P]ATP (Amersham Pharmacia Biotech, Amersham, UK). An assay stock buffer solution was prepared containing all of the reagents listed above, with the exception of ATP, and the test compound of interest. 15 μl of the stock solution was placed in a 96 well plate followed by addition of 1 μl of 0.5 mM DMSO stock containing the test compound (final compound concentration 25 μM, final DMSO concentration 5%). The plate was preincubated for about 10 minutes at 30° C. and the reaction initiated by addition of 4 μl ATP (final concentration 40 μM).

The reaction was stopped after 10 minutes by the addition of 100 μL 100 mM phosphoric acid, 0.01% Tween-20. A phosphocellulose 96 well plate (Millipore, Cat no. MAPHNOB50) was pretreated with 100 μL 100 mM phosphoric acid, 0.01% Tween-20 prior to the addition of the reaction mixture (100 μL). The spots were left to soak for at least 5 minutes, prior to wash steps (4×200 μL 100 mM phosphoric acid, 0.01% Tween-20). After drying, 20 μL Optiphase ‘SuperMix’ liquid scintillation cocktail (Perkin Elmer) was added to the well prior to scintillation counting (1450 Microbeta Liquid Scintillation Counter, Wallac).

Compounds showing greater than 50% inhibition versus standard wells containing the assay mixture and DMSO without test compound were titrated to determine IC₅₀ values.

### Example 3

### ROCK Inhibition Assay

Compounds were screened for their ability to inhibit ROCK using a standard coupled enzyme assay (Fox et al (1998) Protein Sci 7, 2249). Reactions were carried out in 100 mM HEPES pH 7.5, 10 mM MgCl2, 25 mM NaCl, 1 mM DTT and 1.5% DMSO. Final substrate concentrations in the assay were 13 μM ATP (Sigma chemicals) and 200 μM peptide (American Peptide, Sunnyvale, Calif.). Assays were carried out at 30° C. and 200 nM ROCK. Final concentrations of the components of the coupled enzyme system were 2.5 mM phosphoenolpyruvate, 400 μM NADH, 30 μg/ml pyruvate kinase and 10 μg/ml lactate dehydrogenase.

An assay stock buffer solution was prepared containing all of the reagents listed above, with the exception of ROCK, DTT and the test compound of interest. 56 μl of the test reaction was placed in a 384 well plate followed by addition of 1 μl of 2 mM DMSO stock containing the test compound (final compound concentration 30 μM). The plate was preincubated for about 10 minutes at 30° C. and the reaction initiated by addition of 10 μl of enzyme (final concentration 100 nM). Rates of reaction were obtained using a BioRad Ultramark plate reader (Hercules, Calif.) over a 5 minute read time at 30° C. Compounds showing >50 % inhibition versus standard wells containing DMSO, but no compound, were titrated and IC50's determined using a similar protocol.

The following compounds were shown to have Ki values of less than or equal to 2 μM for ROCK: I-3, I-5, I-6, I-8 to I-10, I-19, I-25, I-29, I-31, I-34, I-36 to I-40, I-42, I-44, I-49, I-55 to I-60, I-64, I-68, I-69, I-71, I-72, I-74, I-77 to I-79, I-81, I-83, I-84, I-88, I-90, I-92, I-95, I-97, I-99, I-101, I-102, I-104 to I-108, I-112 to I-114, I-120, I-122, and I-127.

### Example 4

### p70S6K Inhibition Assay

Compounds were screened for their ability to inhibit p70S6K using a radioactive-phosphate incorporation assay at Upstate Biotechnology (Pitt and Lee, J. Biomol. Screen., (1996) 1, 47). Assays were carried out in a mixture of 8 mM MOPS (pH 7.0), 10 mM MgAcetate, 0.2 mM EDTA. Final substrate concentrations in the assay were 15 μM ATP (Sigma Chemicals) and 100 μM peptide (Upstate Ltd., Dundee, UK). Assays were carried out at 30° C. and in the presence of p70S6K (5-10 mU, Upstate Ltd., Dundee, UK) and [γ-³³P]ATP (Specific activity approx. 500 cpm/pmol, Amersham Pharmacia Biotech, Amersham, UK). An assay stock buffer solution was prepared containing all of the reagents listed above, with the exception of ATP, and the test compound of interest. 15 μL of the stock solution was placed in a 96 well plate followed by addition of 1 μL of 40 μM or 8 μM DMSO stock containing the test compound, in duplicate (final compound concentration 2 μM or 0.4 μM, respectively, final DMSO concentration 5%). The plate was preincubated for about 10 minutes at 30° C. and the reaction initiated by addition of 4 μL ATP (final concentration 15 μM).

The reaction was stopped after 10 minutes by the addition of 5 μL 3% phosphoric acid solution. A phosphocellulose 96 well plate (Millipore, Cat no. MAPHNOB50) was pretreated with 100 μL 100 mM phosphoric acid, 0.01% Tween-20 prior to the addition of the reaction mixture (20 μL). The spots were left to soak for at least 5 minutes, prior to wash steps (4×200 μL 100 mM phosphoric acid, 0.01% Tween-20). After drying, 20 μL Optiphase ‘SuperMix’ liquid scintillation cocktail (Perkin Elmer) was added to the well prior to scintillation counting (1450 Microbeta Liquid Scintillation Counter, Wallac).

Percentage inhibition of compounds at 2 μM and 0.4 μM was calculated by comparing p70S6K activity with standard wells containing the assay mixture and DMSO without test compound.

The following compounds were shown to have Ki values of less than or equal to 2 μM for p70S6K: I-3 to I-126, I-130 to I-134, and I-139.

Compounds showing high inhibition versus standard wells were titrated to determine IC₅₀ values.

While we have described a number of embodiments of this invention, it is apparent that our basic examples may be altered to provide other embodiments which utilize the compounds and methods of this invention. Therefore, it will be appreciated that the scope of this invention is to be defined by the appended claims rather than by the specific embodiments which have been represented by way of example.

1. A compound of formula I: 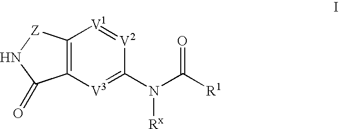
or a pharmaceutically acceptable salt thereof, wherein:
Z is —CH₂— or —C(O)—;
R¹ is selected from T-R, T-Ar, or T-C(R)(T-Ar)R², wherein:
R and R² optionally form a 5-7 membered saturated or partially unsaturated ring having 0-4 heteroatoms independently selected from nitrogen, oxygen, or sulfur;
R^{x} is T-R or T-Ar, or:
R^{x} and R¹ are taken together to form an optionally substituted 5-7 membered saturated or partially unsaturated ring having 1-4 heteroatoms independently selected from nitrogen, oxygen, or sulfur;
each T is independently selected from a valence bond or a C₁₋₆ alkylidene chain, wherein up to two methylene units of T are optionally, and independently, replaced by —O—, —N(R)—, —S—, —N(R)C(O)—, —C(O)N(R)—, —C(O)—, or —SO₂—;
each R is independently selected from hydrogen or an optionally substituted C₁₋₆ aliphatic group, or:
two R groups on the same nitrogen, taken together with the nitrogen atom attached thereto, form a 5-7 membered saturated, partially unsaturated, or fully unsaturated ring having 1-3 heteroatoms independently selected from nitrogen, oxygen, or sulfur;
each Q is independently selected from a valence bond or a C₁₋₄ alkylidene chain;
each Ar is independently an optionally substituted ring selected from a 5-7 membered saturated, partially unsaturated, or fully unsaturated monocyclic ring having 0-4 heteroatoms independently selected from nitrogen, oxygen, or sulfur, or an 8-10 membered saturated, partially unsaturated, or fully unsaturated bicyclic ring having 0-4 heteroatoms independently selected from nitrogen, oxygen, or sulfur;
R² is selected from R′, Ar¹, Q-OR³, Q-OC(O)R³, Q-CONHR⁴, Q-OC(O)NHR⁴, Q-SR³, Q-N(R⁴)₂, N(R)(Q-Ar), N(R)C(O)Q-N(R⁴)₂, or N(R)Q-N(R⁴)₂;
R′ is an optionally substituted C₁₋₆ aliphatic group;
each R³ is independently selected from R or Ar;
each R⁴ is independently selected from R, COR³, CO₂R³, CON(R³)₂, SO₂R³, SO₂N(R³)₂, or Ar¹;
V¹, V² and V³ are each independently selected from nitrogen or C(R⁵);
each R⁵ is independently selected from R, Ar¹, halogen, CN, NO₂, OR, SR, N(R⁴)₂, N(R)COR, N(R)CON(R⁴)₂, N(R)C(O)OR, CON(R⁴)₂, OC(O)N(R⁴)₂, CO₂R, OC(O)R, N(R)SO₂R, N(R)SO₂N(R⁴)₂, SO₂R, or SO₂N(R⁴)₂; and
each Ar¹ is independently selected from an optionally substituted 5-7 membered saturated, partially unsaturated, or fully unsaturated monocyclic ring having 0-4 heteroatoms independently selected from nitrogen, oxygen, or sulfur;
provided that said compound is other than the group consisting of:
N-(2,3-dihydro-3-oxo-1H-isoindol-5-yl)-benzamide,
5-(3,4-dimethylphenoxy)-[2,5′-bi-2H-isoindole]-1,1′,3,3′-tetrone,
N-(2,3-dihydro-1,3-dioxo-1H-isoindol-5-yl)-2-methoxy-benzamide,
N-(2,3-dihydro-1,3-dioxo-1H-isoindol-5-yl)-2-ethoxy-benzamide,
N-(2,3-dihydro-1,3-dioxo-1H-isoindol-5-yl)-2-(3,4-dimethylphenoxy)-acetamide,
N-(2,3-dihydro-1,3-dioxo-1H-isoindol-5-yl)-2-(2,5-dimethylphenoxy)-acetamide,
N-(2,3-dihydro-1,3-dioxo-1H-isoindol-5-yl)-2-(3-methoxyphenoxy)-acetamide,
N-(2,3-dihydro-1,3-dioxo-1H-isoindol-5-yl)-1,3-dihydro-5-nitro-1,3-dioxo-(2H-isoindole-2-acetamide),
N-(2,3-dihydro-1,3-dioxo-1H-isoindol-5-yl)-1,3-dihydro-1,3-dioxo-(2H-isoindole-2-acetamide),
N-(2,3-dihydro-1,3-dioxo-1H-isoindol-5-yl)-2-[4-(1-methylpropyl)phenoxy]-acetamide,
N-(2,3-dihydro-1,3-dioxo-1H-isoindol-5-yl)-2-(4-iodophenoxy)-acetamide,
N-(2,3-dihydro-1,3-dioxo-1H-isoindol-5-yl)-2-(phenylthio)-acetamide,
N-(2,3-dihydro-1,3-dioxo-1H-isoindol-5-yl)-3-(3-nitrophenyl)-2-propenamide,
N-(2,3-dihydro-1,3-dioxo-1H-isoindol-5-yl)-3-phenyl-2-propenamide,
3-(5-bromo-2-methoxyphenyl)-N-(2,3-dihydro-1,3-dioxo-1H-isoindol-5-yl)-2-propenamide,
12-(2,3-dihydro-1,3-dioxo-1H-isoindol-5-yl)-11H-benzo[5,6][1,4]benzodioxocino[2,3-f]isoindole-11,13(12H)-dione,
N-(2,3-dihydro-1,3-dioxo-1H-isoindol-5-yl)-2-phenoxy-propanamide,
2-[(4-chlorophenyl)thio]-N-(2,3-dihydro-1,3-dioxo-1H-isoindol-5-yl)-acetamide,
N-[3-[[(2,3-dihydro-1,3-dioxo-1H-isoindol-5-yl)amino]carbonyl]phenyl]-2-furancarboxamide,
2-(4-bromophenoxy)-N-(2,3-dihydro-1,3-dioxo-1H-isoindol-5-yl)-acetamide,
2-bromo-N-(2,3-dihydro-1,3-dioxo-1H-isoindol-5-yl)-benzamide,
N-(2,3-dihydro-1,3-dioxo-1H-isoindol-5-yl)-2-[4-(1-methylethyl)phenoxy]-acetamide,
2-(2-chlorophenoxy)-N-(2,3-dihydro-1,3-dioxo-1H-isoindol-5-yl)-acetamide,
4-chloro-N-(2,3-dihydro-1,3-dioxo-1H-isoindol-5-yl)-benzeneacetamide,
4-[[bis(2-cyanoethyl)amino]sulfonyl]-N-(2,3-dihydro-1,3-dioxo-1H-isoindol-5-yl)-benzamide,
N-(2,3-dihydro-1,3-dioxo-1H-isoindol-5-yl)-2,3,3,3-tetrafluoro-2-methoxy-propanamide,
N-(2,3-dihydro-1,3-dioxo-1H-isoindol-5-yl)-2,2,2-trifluoro-acetamide,
N-(2,3-dihydro-1,3-dioxo-1H-isoindol-5-yl)-benzeneacetamide,
N-(2,3-dihydro-1,3-dioxo-1H-isoindol-5-yl)-2-furancarboxamide,
N-(2,3-dihydro-1,3-dioxo-1H-isoindol-5-yl)-1,2,3,6-tetrahydro-2,6-dioxo-4-pyrimidinecarboxamide,
N-(2,3-dihydro-1,3-dioxo-1H-isoindol-5-yl)-2-phenoxy-acetamide,
1-(4-chlorophenyl)-N-(2,3-dihydro-1,3-dioxo-1H-isoindol-5-yl)-1,2,5,6,7,8-hexahydro-7,7-dimethyl-2,5-dioxo-3-quinolinecarboxamide,
N-(2,3-dihydro-1,3-dioxo-1H-isoindol-5-yl)-benzenepropanamide,
N-(2,3-dihydro-1,3-dioxo-1H-isoindol-5-yl)-1,2,5,6,7,8-hexahydro-7,7-dimethyl-2,5-dioxo-1-phenyl-3-quinolinecarboxamide,
2-[[(2,4-dichlorophenyl)methyl]thio]-N-(2,3-dihydro-1,3-dioxo-1H-isoindol-5-yl)-acetamide,
4-amino-N-(2,3-dihydro-1,3-dioxo-1H-isoindol-5-yl)-1-methyl-1H-pyrazole-3-carboxamide,
N-(2,3-dihydro-1,3-dioxo-1H-isoindol-5-yl)-1-methyl-4-nitro-1H-pyrazole-3-carboxamide,
4-(acetyloxy)-N-(2,3-dihydro-1,3-dioxo-1H-isoindol-5-yl)-benzamide,
3-chloro-N-(2,3-dihydro-1,3-dioxo-1H-isoindol-5-yl)-benzo[b]thiophene-2-carboxamide,
N-(2,3-dihydro-1,3-dioxo-1H-isoindol-5-yl)-2-thiophenecarboxamide,
N-(2,3-dihydro-1,3-dioxo-1H-isoindol-5-yl)-2-methyl-propanamide,
4-amino-N-(2,3-dihydro-1,3-dioxo-1H-isoindol-5-yl)-benzamide,
3,5-diamino-N-(2,3-dihydro-1,3-dioxo-1H-isoindol-5-yl)-benzamide,
N-(2,3-dihydro-1,3-dioxo-1H-isoindol-5-yl)-cyclohexanecarboxamide,
2,4-dichloro-N-(2,3-dihydro-1,3-dioxo-1H-isoindol-5-yl)-benzamide,
4-[[(2,3-dihydro-1,3-dioxo-1H-isoindol-5-yl)amino]carbonyl]-benzoic acid, methyl ester,
N-(2,3-dihydro-1,3-dioxo-1H-isoindol-5-yl)-4-nitro-benzamide,
N-(2,3-dihydro-1,3-dioxo-1H-isoindol-5-yl)-3,5-dinitro-benzamide,
4-[(diethylamino)sulfonyl]-N-(2,3-dihydro-1,3-dioxo-1H-isoindol-5-yl)-benzamide,
N-(2,3-dihydro-1,3-dioxo-1H-isoindol-5-yl)-1-[(4-methylphenyl)sulfonyl]-cyclopropanecarboxamide,
4-chloro-1H-isoindole-1,3(2H)-dione, 2-(2,3-dihydro-1,3-dioxo-1H-isoindol-5-yl)-4-nitro-[2,5′-Bi-2H-isoindole]-1,1′,3,3′-tetrone,
N-(2,3-dihydro-1,3-dioxo-1H-isoindol-5-yl)-2-methyl-2-propenamide,
N-(2,3-dihydro-1,3-dioxo-1H-isoindol-5-yl)-3-hydroxy-2-naphthalenecarboxamide,
N-(2,3-dihydro-1,3-dioxo-1H-isoindol-5-yl)-4-methoxy-benzamide,
4-chloro-N-(2,3-dihydro-1,3-dioxo-1H-isoindol-5-yl)-benzamide,
N-(2,3-dihydro-1,3-dioxo-1H-isoindol-5-yl)-benzamide,
2-chloro-N-(2,3-dihydro-1,3-dioxo-1H-isoindol-5-yl)-acetamide, and
N-(2,3-dihydro-1,3-dioxo-1H-isoindol-5-yl)-acetamide.

2. The compound according to claim 1, wherein said compound is of formula Ia: 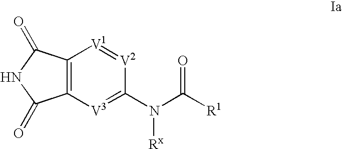
or a pharmaceutically acceptable salt thereof.

3. The compound according to claim 2, wherein R^{x} is hydrogen or T-Ar.

4. The compound according to claim 2, wherein R^{x} and R¹ are taken together to form an optionally substituted 5-7 membered saturated or partially unsaturated ring having 1-4 heteroatoms independently selected from nitrogen, oxygen, or sulfur.

5. The compound according to claim 2, wherein V¹ is CH, V² is CH, and V³ is CH.

6. The compound according to claim 5, wherein:
R¹ is T-Ar; and
Ar is an optionally substituted ring selected from a 5-6 membered saturated, partially unsaturated, or fully unsaturated monocyclic ring having 0-4 heteroatoms independently selected from nitrogen, oxygen, or sulfur, or a 9-10 membered saturated, partially unsaturated, or fully unsaturated bicyclic ring having 0-4 heteroatoms independently selected from nitrogen, oxygen, or sulfur.

7. The compound according to claim 6, wherein:
R¹ is T-Ar; and
Ar is an optionally substituted ring selected from phenyl, pyridyl, pyrimidinyl, pyridonyl, furanyl, tetrazolyl, thienyl, cyclopentyl, cyclohexyl, cycloheptyl, benzo[1,3]dioxolyl, indan-1-onyl, naphthyl, benzothiophenyl, 2,3-dihydro-1H-isoindolyl, indanyl, benzofuranyl, or indolyl.

8. The compound according to claim 5, wherein:
R¹ is T-C(R)(T-Ar)R²; and
R² is selected from R′, Q-OR³, Q-N(R⁴)₂, Ar¹, N(R)C(O)Q-N(R⁴)₂, or N(R)Q-N(R⁴)₂.

9. The compound according to claim 8, wherein Ar is an optionally substituted 5-6 membered saturated, partially unsaturated, or fully unsaturated monocyclic ring having 0-4 heteroatoms independently selected from nitrogen, oxygen, or sulfur, or an optionally substituted 9-10 membered saturated, partially unsaturated, or fully unsaturated bicyclic ring having 0-4 heteroatoms independently selected from nitrogen, oxygen, or sulfur.

10. The compound according to claim 1, wherein said compound is of formula Ib: 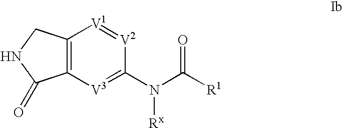
or a pharmaceutically acceptable salt thereof.

11. The compound according to claim 10, wherein R^{x} is hydrogen or T-Ar.

12. The compound according to claim 10, wherein R^{x} and R¹ are taken together to form an optionally substituted 5-7 membered saturated or partially unsaturated ring having 1-4 heteroatoms independently selected from nitrogen, oxygen, or sulfur.

13. The compound according to claim 10, wherein V¹ is CH, V² is CH, and V³ is CH.

14. The compound according to claim 13, wherein:
R¹ is T-Ar; and
Ar is an optionally substituted ring selected from a 5-6 membered saturated, partially unsaturated, or fully unsaturated monocyclic ring having 0-4 heteroatoms independently selected from nitrogen, oxygen, or sulfur, or a 9-10 membered saturated, partially unsaturated, or fully unsaturated bicyclic ring having 0-4 heteroatoms independently selected from nitrogen, oxygen, or sulfur.

15. The compound according to claim 14, wherein:
R¹ is T-Ar; and
Ar is an optionally substituted ring selected from phenyl, pyridyl, pyrimidinyl, pyridonyl, furanyl, tetrazolyl, thienyl, cyclopentyl, cyclohexyl, cycloheptyl, benzo[1,3]dioxolyl, indan-1-onyl, naphthyl, benzothiophenyl, 2,3-dihydro-1H-isoindolyl, indanyl, benzofuranyl, or indolyl.

16. The compound according to claim 13, wherein:
R¹ is T-C(R)(T-Ar)R²; and
R² is selected from R′, Q-OR³, Q-N(R⁴)₂, Ar¹, N(R)C(O)Q-N(R⁴)₂, or N(R)Q-N(R⁴)₂.

17. The compound according to claim 16, wherein Ar is an optionally substituted 5-6 membered saturated, partially unsaturated, or fully unsaturated monocyclic ring having 0-4 heteroatoms independently selected from nitrogen, oxygen, or sulfur, or an optionally substituted 9-10 membered saturated, partially unsaturated, or fully unsaturated bicyclic ring having 0-4 heteroatoms independently selected from nitrogen, oxygen, or sulfur.

18. A composition comprising a compound according to claim 1, and a pharmaceutically acceptable carrier, adjuvant, or vehicle.

19. The composition according to claim 18, wherein said compound is in an amount sufficient to detectably inhibit AKT, PDK1, p70S6k, or ROCK protein kinase activity.

20. The composition according to claim 18, additionally comprising a therapeutic agent selected from a chemotherapeutic or anti-proliferative agent, an anti-inflammatory agent, an immunomodulatory or immunosuppressive agent, a neurotrophic factor, an agent for treating cardiovascular disease, an agent for treating destructive bone disorders, an agent for treating liver disease, an anti-viral agent, an agent for treating blood disorders, an agent for treating diabetes, or an agent for treating immunodeficiency disorders.

21. A method of inhibiting AKT, PDK1, p70S6k, or ROCK kinase activity in:
(a) a patient; or
(b) a biological sample;
which method comprises administering to said patient, or contacting said biological sample with:
a) a composition according to claim 18; or
b) a compound according to claim 1.

22. A method of treating or lessening the severity of a cancer or a proliferative disorder in a patient in need thereof, comprising the step of administering to said patient:
a) a composition according to claim 18; or
b) a compound according to claim 1.

23. The method according to claim 22, comprising the additional step of administering to said patient an additional therapeutic agent selected from a chemotherapeutic or anti-proliferative agent, wherein said additional therapeutic agent is administered together with said composition as a single dosage form or separately from said composition as part of a multiple dosage form.

24. A method of treating or lessening the severity of tuberous sclerosis, or a cancer selected from brain (gliomas), breast, colon, head and neck, kidney, lung, liver, melanoma, ovarian, pancreatic, prostate, sarcoma, or thyroid, in a patient in need thereof, comprising administering to said patient a composition according to claim 18.

25. The method according to claim 24, wherein said disease or condition is selected from pancreatic, prostate, or ovarian cancer.

26. A method of treating or lessening the severity of rheumatoid arthritis, asthma, HIV, angina pectoris, peripheral circulation disorder, ischemia/reperfusion, hypertension, arteriosclerosis, osteoporosis, or benign prostatic hyperplasia, in a patient in need thereof, comprising administering to said patient a composition according to claim 18.

27. A method of treating an autoimmune disorder in a patient in need thereof, comprising administering to said patient a composition according to claim 18.

28. A composition comprising an effective amount of a compound of formula I: 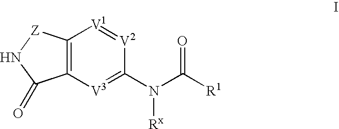
or a pharmaceutically acceptable salt thereof, wherein:
Z is —CH₂— or —C(O)—;
R¹ is selected from T-R, T-Ar, or T-C(R)(T-Ar)R², wherein:
R and R² optionally form a 5-7 membered saturated or partially unsaturated ring having 0-4 heteroatoms independently selected from nitrogen, oxygen, or sulfur;
R^{x} is T-R or T-Ar, or:
R^{x} and R¹ are taken together to form an optionally substituted 5-7 membered saturated or partially unsaturated ring having 1-4 heteroatoms independently selected from nitrogen, oxygen, or sulfur;
each T is independently selected from a valence bond or a C₁₋₆ alkylidene chain, wherein up to two methylene units of T are optionally, and independently, replaced by —O—, —N(R)—, —S—, —N(R)C(O)—, —C(O)N(R)—, —C(O)—, or —SO₂—;
each R is independently selected from hydrogen or an optionally substituted C₁₋₆ aliphatic group, or:
two R groups on the same nitrogen, taken together with the nitrogen atom attached thereto, form a 5-7 membered saturated, partially unsaturated, or fully unsaturated ring having 1-3 heteroatoms independently selected from nitrogen, oxygen, or sulfur;
each Q is independently selected from a valence bond or a C₁₋₄ alkylidene chain;
each Ar is independently an optionally substituted ring selected from a 5-7 membered saturated, partially unsaturated, or fully unsaturated monocyclic ring having 0-4 heteroatoms independently selected from nitrogen, oxygen, or sulfur, or an 8-10 membered saturated, partially unsaturated, or fully unsaturated bicyclic ring having 0-4 heteroatoms independently selected from nitrogen, oxygen, or sulfur;
R² is selected from R′, Ar¹, Q-OR³, Q-OC(O)R³, Q-CONHR⁴, Q-OC(O)NHR⁴, Q-SR³, Q-N(R⁴)₂, N(R)(Q-Ar), N(R)C(O)Q-N(R⁴)₂, or N(R)Q-N(R⁴)₂;
R′ is an optionally substituted C₁₋₆ aliphatic group;
each R³ is independently selected from R or Ar;
each R⁴ is independently selected from R, COR³, CO₂R³, CON(R³)₂, SO₂R³, SO₂N(R³)₂, or Ar¹;
V¹, V² and V³ are each independently selected from nitrogen or C(R⁵);
each R⁵ is independently selected from R, Ar¹, halogen, CN, NO₂, OR, SR, N(R⁴)₂, N(R)COR, N(R)CON(R⁴)₂, N(R)C(O)OR, CON(R⁴)₂, OC(O)N(R⁴)₂, CO₂R, OC(O)R, N(R)SO₂R, N(R)SO₂N(R⁴)₂, SO₂R, or SO₂N(l⁴)₂; and
each Ar¹ is independently selected from an optionally substituted 5-7 membered saturated, partially unsaturated, or fully unsaturated monocyclic ring having 0-4 heteroatoms independently selected from nitrogen, oxygen, or sulfur,
and a pharmaceutically acceptable carrier, adjuvant, or vehicle.

29. The composition according to claim 28, wherein said compound is in an amount sufficient to detectably inhibit AKT, PDK1, p70S6k, or ROCK protein kinase activity.

30. The composition according to claim 28, additionally comprising a therapeutic agent selected from a chemotherapeutic or anti-proliferative agent, an anti-inflammatory agent, an immunomodulatory or immunosuppressive agent, a neurotrophic factor, an agent for treating cardiovascular disease, an agent for treating destructive bone disorders, an agent for treating liver disease, an anti-viral agent, an agent for treating blood disorders, an agent for treating diabetes, or an agent for treating immunodeficiency disorders.

31. A method of inhibiting AKT, PDK1, p70S6k, or ROCK kinase activity in:
(a) a patient; or
(b) a biological sample;
which method comprises administering to said patient, or contacting said biological sample with a composition according to claim 28.

32. A method of treating or lessening the severity of a cancer or a proliferative disorder in a patient in need thereof, comprising the step of administering to said patient a composition according to claim 28.

33. The method according to claim 32, comprising the additional step of administering to said patient an additional therapeutic agent selected from a chemotherapeutic or anti-proliferative agent, wherein said additional therapeutic agent is administered together with said composition as a single dosage form or separately from said composition as part of a multiple dosage form.

34. A method of treating or lessening the severity of tuberous sclerosis, or a cancer selected from brain (gliomas), breast, colon, head and neck, kidney, lung, liver, melanoma, ovarian, pancreatic, prostate, sarcoma, or thyroid, in a patient in need thereof, comprising administering to said patient a composition according to claim 28.

35. The method according to claim 34, wherein said disease or condition is selected from pancreatic, prostate, or ovarian cancer.

36. A method of treating or lessening the severity of rheumatoid arthritis, asthma, HIV, angina pectoris, peripheral circulation disorder, ischemia/reperfusion, hypertension, arteriosclerosis, osteoporosis, or benign prostatic hyperplasia in a patient in need thereof, comprising administering to said patient a composition according to claim 28.

37. A method of treating an autoimmune disorder in a patient in need thereof, comprising administering to said patient a composition according to claim 28.
